(19) United States
(12) Patent Application Publication
(10) Pub. No.: US 2005/0153872 A1
(43) Pub. Date: Jul. 14, 2005

(54) Methods and compositions for the treatment of infection

(76) Inventor: Xiao-Qing Qiu, Hamburg, PA (US)

Correspondence Address: LAHIVE & COCKFIELD, LLP., 28 STATE STREET, BOSTON, MA, 02109, US

(21) Appl. No.: 10/826,132

(22) Filed: Apr. 16, 2004

Related U.S. Application Data
(60) Provisional application No. 60/553,716, filed on Mar. 15, 2004.
(60) Provisional application No. 60/559,232, filed on Apr. 2, 2004.
(60) Provisional application No. 60/535,767, filed on Jan. 12, 2004.

(51) Int. Cl. A61K 38/16; C07K 14/195; C07K 16/28; C07K 14/32

(52) U.S. Cl. 514/2; 530/350; 530/388.22; 424/144.1

(57) ABSTRACT

The invention described herein provides novel compositions and methods for treating infection caused by, for example, bacteria, fungi, or virus.

### BACKGROUND

The worldwide use of antibiotics to treat infectious diseases in humans and animals has grown dramatically over the last forty years. In 1954, two million pounds of antibiotics were produced in the United States. Today, the figure exceeds 50 million pounds. According to the Centers Disease Control (CDC), humans consume 235 million doses of antibiotics annually. Widespread misuse or overuse of antibiotics has fostered the spread of antibiotic resistance and has contributed to the development of a serious public health problem. For example, the bacterium Staphylococcus aureus is a major cause of hospital acquired infections that, historically, responded satisfactorily to the antibiotic vancomycin. Recently, however, many strains of S. aureus have been found to be resistant to vancomycin. Moreover, the death rate for some communicable diseases such as tuberculosis have started to rise again, in part because of increases in bacterial resistance to antibiotics.

The occurrence of antibiotic resistance in bacteria has spurred the search for new antibacterial compounds. In large part, this search for new drugs has focused on identifying the defense molecules that plants and animals themselves produce to fight infections. One group of compounds that has gained researchers' attention are the peptide antibiotics. The benefit from use of antibiotics as a means of treating infections has been increasingly compromised by the development of resistant strains of microorganisms. Most of the new drugs are derivatives of older compounds.

Accordingly, it is necessary to develop new agents that will respond to the current needs for compounds that will effectively control pathogenic populations.

### SUMMARY OF THE INVENTION

The instant invention is based on the discovery that a bifunctional molecule comprising an organism targeting agent and a channel forming moiety, can selectively kill microorganisms, fungi, and viral infected cells. The invention provides a novel class of compounds that kill organisms or cells infected by an organism, e.g., viral infected cells, with a mechanism different from all currently available compounds. Accordingly, in one aspect, the instant invention provides a molecule comprising an organism targeting agent covalently attached to a channel forming moiety. In one embodiment, the molecule is a polypeptide.

In certain embodiments, the channel forming moiety is a channel forming polypeptide, a channel forming domain or fragment thereof. For example, the channel forming polypeptide can be α-hemolysin, delta toxin, anthrax toxin, and colicin. In particular embodiments the colicin is E1, Ia, Ib, A, B or N. In one particular embodiment, the colicin is colicin Ia.

In a related embodiment, the channel forming fragment is a fragment of colicin comprising amino acid residues from about 544 to about amino acid residue 626 or amino acid residues from about 524 to about amino acid residue 626 of colicin Ia. In a specific embodiment, the channel forming domain comprises amino acid residues 544-626.

In specific embodiments, the organism targeting agent is selected from the group consisting of a ligand, an antibody, an antibody fragment, a reconstituted antibody mimetic, a phage segment, and a pheromone.

In specific embodiments the organism targeting agent is a pheromone. In related embodiments, the pheromone is from an organism selected from the group consisting of: Staphylococcus, Enterococcus, and Streptococcus.

In specific embodiments the pheromone is Staphylococcus aureus pheromone is AgrDI, AgrDII, AgrDIII or AgrDIV.

In another specific embodiment, the pheromone is Streptococcus pneumoniae pheromone CSP.

In another embodiment, the pheromone is Enterococcus fecaelis pheromone cCF10.

In one embodiment the organism targeting agent is C-terminal to the channel forming moiety. In an alternative embodiment, the organism targeting agent is N-terminal to the channel forming moiety.

In another embodiment, the organism targeting agent is an antibody or fragment thereof, or a reconstituted antibody mimetic.

In a related embodiment, the antibody, a fragment thereof, or a reconstituted antibody mimetic is specific for a polypeptide expressed by an organism, e.g., a virus, fungus, or bacteria. In a related embodiment, the antibody is an ScFv or a reconstituted antibody mimetic.

In specific embodiments, the ScFv, or a reconstituted antibody mimetic, recognizes Hepatitis virus B PreS1 antigen, or hepatitis virus B HBsAg antigen.

In one aspect, the invention provides a polypeptide comprising the Staphylococcus aureus pheromone AgrDI and a channel forming domain, or fragment thereof, of colicin. In a related embodiment, the channel forming domain of colicin comprises residues 524-626 of colicin Ia. In a specific embodiment, the channel forming domain comprises amino acid residues 544-626.

In one aspect, the invention provides a polypeptide comprising the Streptococcus pneumoniae pheromone CSP and a channel forming moiety, a channel forming domain thereof, or fragment thereof, of colicin. In a specific embodiment, the channel forming domain of colicin comprises residues 524-626 of colicin Ia. In a specific embodiment, the channel forming domain comprises amino acid residues 544-626.

In one aspect, the invention provides a polypeptide comprising the Enterococcus fecaelis pheromone cCF10 and a channel forming moiety, a channel forming domain, or fragment thereof of colicin. In a specific embodiment, the channel forming domain of colicin comprises residues from about residue 524 to about residue 626 of colicin Ia. In a specific embodiment, the channel forming domain comprises amino acid residues 544-626.

In one aspect, the invention provides a polypeptide comprising the HBV PreS1 ScFv, or a reconstituted antibody mimetic, and a channel forming domain of colicin. In a specific embodiment, the channel forming domain of colicin comprises residues from about residue 524 to about residue 626 of colicin Ia. In a specific embodiment, the channel forming domain comprises amino acid residues 544-626.

In one aspect, the invention provides a polypeptide comprising the HBV HBsAg ScFv and a channel forming domain of colicin. In a specific embodiment, the channel forming domain of colicin comprises residues from about residue 524 to about residue 626 of colicin Ia. In a specific embodiment, the channel forming domain comprises amino acid residues 544-626.

In one aspect, the invention provides a polypeptide comprising the Candida Albicans mating pheromone and a channel forming moiety, channel forming domain, or fragment thereof of colicin. In a specific embodiment, the channel forming domain of colicin comprises residues from about residue 524 to about residue 626 of colicin Ia. In a specific embodiment, the channel forming domain comprises amino acid residues 544-626.

In a related embodiment, the polypeptides of the invention may have one or more non natural amino acid residues, e.g., amino acid analogs, or mimetics.

In a specific embodiment, the non-natural amino acid residues are D-isomers of natural amino acid residues.

In another aspect, the invention provides a nucleic acid molecule that encodes a polypeptides of the invention.

In a related embodiment, the invention provides a vector, e.g., an expression vector, comprising a nucleic acid molecule that encodes a polypeptide of the invention.

In a related embodiment, the invention provides a host cell comprising a vector of the invention. In specific embodiments, the host cell is a bacterial host cell, e.g., E. Coli, or a mammalian host cell.

In another aspect, the invention further provides methods of producing the a polypeptides of the invention. The methods comprise the steps of culturing the host cells of the invention such that the polypeptides are produced. In a further embodiment, the methods of the invention may involve purifying said polypeptide.

In another aspect, the invention provides a method of producing a molecule of the invention wherein the organism targeting agent and the channel forming moiety are produced separately and covalently linked after production. In a related embodiment, the channel forming moiety and/or the organism targeting agent are produced recombinantly.

In another aspect, the invention provides methods of treating a subject having a bacterial infection, comprising administering to the subject an effective amount of a polypeptide comprising an organism targeting agent and a channel forming moiety thereby treating the subject.

In a related embodiment, the organism targeting agent is selected from the group consisting of an antibody, a fragment thereof, an ScFV, a reconstituted antibody mimetic, and a pheromone. In a specific embodiment, the pheromone is a bacterial pheromone, e.g., a pheromone from Staphylococcus, Enterococcus or Streptococcus. In specific embodiments, the bacterial pheromone is selected from the group consisting of AgrD I, AgrD II, AgrD III, AgrD IV, cCF10, and CSP. In other specific embodiments, the reconstituted antibody mimetic or ScFV is specific for HBV PreS1 or HBV HBsAg.

In one embodiment, the channel forming moiety is selected from the group consisting of α-hemolysin, delta toxin, diphtheria toxin, anthrax toxin, and colicin, or a fragment thereof. In specific embodiments the colicin, or fragment of colicin, is selected from the group consisting of colicin. E1, Ia, Ib, A, B and N. In one particular embodiment, the colicin is colicin Ia. In further particular embodiments, the fragment of colicin Ia comprises amino acid residues 544-626 or 524-626.

In another aspect, the invention provides a method of treating a subject having a fungal infection by administering to the subject an effective amount of a polypeptide comprising an organism targeting agent and a channel forming moiety, thereby treating the subject.

In a related embodiment, the organism targeting agent is selected from an antibody, a fragment thereof, an ScFv, a reconstituted antibody mimetic and a pheromone. In a specific embodiment, the organism targeting agent is a pheromone, e.g., the C. albicans alpha mating pheromone.

In one embodiment, the channel forming moiety is selected from the group consisting of a-hemolysin, delta toxin, anthrax toxin, and colicin, or a fragment thereof. In a related embodiment, the colicin or fragment of colicin is selected from the group consisting of colicin E1, Ia, Ib, A, B and N. In a specific embodiment, the colicin is colicin Ia. In a further specific embodiment, the fragment of colicin is selected from the group consisting of amino acid residues 544-626 and 524-626 of colicin Ia.

In another aspect, the invention provides a method of treating a subject having a viral infection comprising administering to the subject an effective amount of a polypeptide comprising an organism targeting agent and a channel forming moiety thereby treating said subject.

In a one embodiment, the organism targeting agent is selected from the group consisting of an antibody, a fragment thereof, a reconstituted antibody mimetic, and a ScFv. In specific embodiments the ScFv is HBV PreS1 or HBV HBsAg.

In another embodiment, the channel forming moiety is selected from the group consisting of a-hemolysin, delta toxin, anthrax toxin, and colicin, or a fragment thereof. In specific embodiments the colicin is selected from the group consisting of E1, Ia, Ib, A, B and N. In a specific embodiment, the colicin is colicin Ia. In a further specific embodiment, the fragment of colicin Ia is selected from the group consisting of amino acid residues from about residue 544 to about residue 626 and from about 524 to about residue 626. In a specific embodiment, the channel forming domain comprises amino acid residues 544-626.

### BRIEF DESCRIPTION OF THE DRAWINGS

FIG. 1 depicts a schematic representation of vectors comprising molecules of the invention. FIG. 1A depicts a pSELECT™-1 vector comprising a nucleic acid molecule encoding a polypeptide comprising Colicin Ia linked to the N-terminus of the S. aureus pheromone AgrDI. The polypeptide sequence is set forth as SEQ ID NO:1 and the encoding nucleic acid molecule is set forth as SEQ ID NO: 2. FIG. 1B depicts a pSELECT™-1 vector comprising a nucleic acid molecule encoding a polypeptide comprising Colicin Ia linked to the N-terminus of the S. aureus pheromone AgrDII. The polypeptide sequence is set forth as SEQ ID NO:3 and the encoding nucleic acid molecule is set forth as SEQ ID NO: 4. FIG. 1C depicts a pSELECT™-1 vector comprising a nucleic acid molecule encoding a polypeptide comprising Colicin Ia linked to the N-terminus of the S. aureus pheromone AgrDIII. The polypeptide sequence is set forth as SEQ ID NO:5 and the encoding nucleic acid molecule is set forth as SEQ ID NO: 6. FIG. 1D depicts a pSELECT™-1 vector comprising a nucleic acid molecule encoding a polypeptide comprising Colicin Ia linked to the N-terminus of the S. aureus pheromone AgrDIV. The polypeptide sequence is set forth as SEQ ID NO:7 and the encoding nucleic acid molecule is set forth as SEQ ID NO: 8. FIG. 1E depicts a pSELECT™-1 vector comprising a nucleic acid molecule encoding a polypeptide comprising Colicin Ia linked to the N-terminus of the S. epidermis pheromone. The polypeptide sequence is set forth as SEQ ID NO:9 and the encoding nucleic acid molecule is set forth as SEQ ID NO: 10). FIG. 1F depicts a pSELECT™-1 vector comprising a nucleic acid molecule encoding a polypeptide comprising the S. aureus pheromone AgrDI linked to the N-terminus of Colicin Ia. The polypeptide sequence is set forth as SEQ ID NO:11 and the encoding nucleic acid molecule is set forth as SEQ ID NO: 12.

FIG. 2 depicts a graph representing the growth of non-antibiotic resistant S. aureus as a function of time (in hours) after the addition of potential antibacterial agents. Penicillin (PEN), wild-type colicin Ia (COL), TG1 protein (TG1), Colicin Ia linked to a non-specific octomeric peptide (fusion protein), and Colicin Ia linked to the AgrDI pheromone of S. aureus (PMC).

FIG. 3 depicts electron micrographs of methicillin resistant S. aureus strain ATCC BAA-42 (MRSA) treated with (a) control, (b) wild type colicin for 1.5 hours, (c) ArgDI for 1.5 hours, (d) oxacillin for 1.5 hours, and (e) and (f) Colicin Ia linked to the AgrDI pheromone of S. aureus for 0.5 hours.

FIG. 4 depicts the cumulative survival of mice infected with MRSA strain BAA-42 and treated with Colicin Ia linked to the AgrDI pheromone of S. aureus (Ph-SA), vancomycin, penicillin, ampicillin, or cephazoid.

FIGS. 5A-B depict the cumulative survival of mice infected with methicillin resistant Staphylococcus aureus strain ATCC BAA-42 and treated with Colicin Ia linked to the AgrDI pheromone of S. aureus (Ph-SA) or vancomycin. FIG. 5A depicts treatment of mice with ˜5 mg/kg of Ph-SA or vancomycin, and FIG. 5B treatment of mice with ˜1.4 mg/kg of Ph-SA or vancomycin.

FIG. 6 depicts a graph representing the growth of non-antibiotic resistant S. epidermidis as a function of time (in hours) after the addition of potential antibacterial agents. Cells were treated with either penicillin (PEN), Colicin Ia linked to the AgrDI pheromone of S. aureus (Ph-SA), Colicin Ia linked to the pheromone of S. epidermis (Ph-SE) or a control. The ph-SE polypeptide sequence is set forth as SEQ ID NO:13 and the encoding nucleic acid molecule is set forth as SEQ ID NO: 14.

FIG. 7 depicts a graph representing the growth of methicillin resistant S. aureus strain ATCC BAA-42. Cell cultures were treated with Colicin Ia linked to the AgrDI pheromone of S. aureus (Ph-SA1), Colicin Ia linked to the AgrDII pheromone of S. aureus (Ph-SA2), Colicin Ia linked to the AgrDIII pheromone of S. aureus (Ph-SA3), Colicin Ia linked to the AgrDIV pheromone of S. aureus (Ph-SA4), the AgrDI pheromone of S. aureus linked to Colicin Ia (Ph-SAR), Colicin Ia linked to the pheromone of S. epidermidis (Ph-SE)or oxacillin. Optical density measurements were collected at 1 hour intervals and graphed versus time of cell growth.

FIG. 8 depicts a pSELECT™-1 vector comprising a nucleic acid molecule encoding a polypeptide comprising Colicin Ia linked to the N-terminus of the S. pneumoniae pheromone Corn C. The polypeptide sequence is set forth as SEQ ID NO:15 and the encoding nucleic acid molecule is set forth as SEQ ID NO: 16.

FIG. 9 depicts a schematic representation of a polypeptide comprising a colicin Ia and the Com C pheromone of S. pneumoniae.

FIG. 10 depicts a graph representing growth of penicillin resistant S. pneumoniae ATCC 70761 as a function of time after treatment with a control (Control), Colicin Ia linked to ComC pheromone of S. pneumoniae (Ph-SP), or penicillin (PEN).

FIG. 11 depicts a graph representing growth of non-resistant S. pneumoniae BQIOC 31201 as a function of time after treatment with a control (Control), wild-type colicin Ia (Col), Colicin Ia linked to Corn C pheromone of S. pneumoniae (Ph-SP), or penicillin (PEN).

FIG. 12 depicts a pSELECT™-1 vector comprising a nucleic acid molecule encoding a polypeptide comprising Colicin Ia linked to the N-terminus of the E. facaelis pheromone cCF10. The polypeptide sequence is set forth as SEQ ID NO:17 and the encoding nucleic acid molecule is set forth as SEQ ID NO:18.

FIG. 13 depicts a schematic representation of a polypeptide comprising a colicin Ia and the cCF10 pheromone of E. facaelis (Ph-EF).

FIG. 14 depicts a graph representing the growth of vancomycin-sensitive E. facaelis ATCC 29212 as a function of time after treatment with a control, Colicin Ia linked to the N-terminus of the E. facaelis pheromone cCF10 (Ph-EF), or penicillin (Pen).

FIG. 15 depicts a graph representing the growth of vancomycin-resistant E. facaelis ATCC 700802 as a function of time after treatment with a control, Colicin Ia linked to the N-terminus of the E. facaelis pheromone cCF10 (Ph-EF), or vancomycin.

FIGS. 16A-H depict electron micrographs of bacterial cells treated with ph-EF, ph-SA, or vancomycin. FIG. 16A depicts untreated vancomycin resistant E. facaelis ATCC 700802. FIGS. 16B and C depict vancomycin resistant E. facaelis ATCC 700802 treated with 10 ug/ml Ph-EF for 0.5 hours. FIG. 16D depicts untreated vancomycin resistant E. facaelis ATCC 700802 after 2.0 hours. FIG. 16E depicts vancomycin resistant E. facaelis ATCC 700802 treated with 10 ug/ml Ph-SA for 2.0 hours. FIG. 16F depicts vancomycin resistant E. facaelis ATCC 700802 treated with 20 ug/ml vancomycin for 2.0 hours. FIGS. 16G and H depict vancomycin resistant E. facaelis ATCC 700802 treated with 10 ug/ml Ph-EF for 2.0 hours.

FIGS. 17A-B depict electron micrograph images of Hepatitis B virus. FIG. 17A depicts untreated HBV. FIG. 17B depicts HBV after one hour incubation with a peptide comprising G28-V50/E217-S228 from HBsAg ScFv attached to colicin Ia. The polypeptide sequence of G28-V50/E217-S228 from HBsAg ScFv attached to colicin Ia is set forth as SEQ ID NO:19 and the encoding nucleic acid molecule is set forth as SEQ ID NO: 20.

FIG. 18A-D depict flasks containing C. albicans ATCC 10231. FIG. 18A depicts C. albicans grown for 22 hours in borate stock solution. FIG. 18B depicts C. albicans grown for 22 hours after treatment with 3 ug/ml fluconazole. FIG. 18C depicts C. albicans grown for 22 hours after treatment with 200 ng/ml amphotericinB. FIG. 18D depicts C. albicans grown for 22 hours after treatment with 5 ug/ml of a fusion protein of colicin Ia and the C. albicans mating pheromone (Ph-CA). The polypeptide sequence of Ph-CA is set forth as SEQ ID NO:21 and the encoding nucleic acid molecule is set forth as SEQ ID NO:22.

FIGS. 19A-D depict flasks containing C. albicans ATCC 10231. FIG. 19A depicts C. albicans grown for 62 hours in borate stock solution. FIG. 19B depicts C. albicans grown for 62 hours after treatment with 50 ug/ml ampicillin. FIG. 19C depicts C. albicans grown for 62 hours after treatment with 5 ug/ml ph-SA. FIG. 19D depicts C. albicans grown for 62 hours after treatment with 5 ug/ml of a fusion protein of colicin Ia and the C. albicans mating pheromone (Ph-CA).

FIGS. 20A-D depict optical microscope views of C. albicans cultures. FIG. 20A depicts a C. albicans culture observed by optical microscope in contrast image mode at 400×. FIG. 20B depicts a C. albicans culture after treatment with 50 ug/ml ampicillin observed by optical microscope in contrast image mode at 400×. FIG. 20C depicts a C. albicans culture after treatment with 5 ug/ml Ph-SA observed by optical microscope in contrast image mode at 400×. FIG. 20D depicts a C. albicans culture after treatment with 5 ug/ml Ph-CA observed by optical microscope in contrast image mode at 400×.

FIGS. 21A-D depict flasks containing A. niger SWA-1011. FIG. 21A depicts A. niger grown for 48 hours in borate stock solution FIG. 21B depicts A. niger grown for 48 hours after treatment with 200 ng/ml amphotericin B. FIG. 21C depicts A. niger grown for 48 hours after treatment with 3 ug/ml fluconazole. FIG. 21D depicts A. niger grown for 48 hours after treatment with 5 ug/ml of a fusion protein of colicin Ia and the C. albicans mating pheromone (Ph-CA).

### DETAILED DESCRIPTION

Before further description of the invention, certain terms employed in the specification, examples and appended claims are, for convenience, collected here.

The term “organism targeting agent” is intended to include molecules, e.g., small molecules, peptides, and polypeptides, that specifically recognize a given type of organism. These agents can either bind to a receptor expressed on the surface of a cell expressed by the organism, or alternatively, bind to a polypeptide or carbohydrate presented on the surface of the organism. Organism targeting agents can be specific for classes of organisms, e.g., fungi, bacteria, or viruses, or for specific genuses of organisms, e.g., Staphylococcus, Enterococcus, herpes virus, or Candida. Organism targeting agents of the invention can also target a specific species of organism, e.g., Staphylococcus aureus as opposed to Staphylococcus epidermidis. The organism targeting agents of the invention can be, for example, without limitation, pheromones, antibodies, reconstituted antibody mimetics, fragments of antibodies, single chain antibodies, or small molecules.

The term “channel forming moiety” is intended to include transmembrane polypeptides that are capable of inserting into a lipid bilayer thereby creating a passageway, i.e, a gating passageway, from inside of the cell compartment to outside of the cell compartment. In preferred embodiments, the passageway is not specific in terms of what is permitted to pass through the channel. Exemplary channel forming moieties are polypeptides that naturally form channels in lipid bilayers, e.g., α-hemolysin, diphtheria toxin, delta toxin, anthrax toxin, and colicin. Channel forming moieties can also be fragments of naturally occurring polypeptides that retain the ability to insert into a lipid bilayer and form a channel. One of skill in the art would be able to isolate many fragments of naturally occurring polypeptides that have the ability to form a channel. Those channel forming fragments are intended to be used in the methods and compositions of the instant invention.

The term “pheromone” is intended to include chemical substances, e.g., small molecules or peptides, that are produced by an organism and dispersed into their surroundings to induce one or more behavioral responses from other organisms. The organisms which are induced into biological response can be the same species or other species. Non-limiting examples of pheromones are bacterial pheromones such as those found in, for example, S. aureus (AgrDI-IV) or S. pneumoniae (ComC).

The term “antibody” as used herein refers to immunoglobulin molecules and immunologically active portions of immunoglobulin molecules, i.e., molecules that contain an antigen binding site which specifically binds (immunoreacts with) an antigen. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope. A monoclonal antibody composition thus typically displays a single binding affinity for a particular protein with which it immunoreacts.

The term “reconstituted antibody mimetic” refers to an organism targeting agent comprising one or more antibody complementarity determining regions (CDRs) covalently linked by, for example, a polypeptide linker. For example, 1, 2, 3, 4, 5, 6, 7, 8, 9, 10, 11, or 12 CDR regions from light or heavy chains may be covalently attached to confer binding affinity, i.e. sufficient binding affinity to allow the molecules of the invention to target an organism and allow for insertion of the channel forming domain to insert into the lipid bilayer. The CDRs may be linked together directly, by using random peptides, or by using the natural linking peptides present in antibody heavy or light chains. The CDR regions are generally from an antibody that specifically recognizes an epitope presented by a target organism.

The terms “protein” and “polypeptide” are used interchangeably herein. The term “peptide” is used herein to refer to a chain of two or more amino acids or amino acid analogs (including non-naturally occurring amino acids), with adjacent amino acids joined by peptide (—NHCO—) bonds. Thus, peptides in accordance with the invention include oligopeptides, polypeptides, proteins, and peptidomimetics.

The term “fragment” refers to any portion of a natural, recombinant or synthetic polypeptide. A fragment can be made synthetically, enzymatically, or recombinantly.

The term “pharmaceutical composition” includes preparations suitable for administration to mammals, e.g., humans. When the compounds of the present invention are administered as pharmaceuticals to mammals, e.g., humans, they can be given per se or as a pharmaceutical composition containing, for example, 0.1 to 99.5% of active ingredient in combination with a pharmaceutically acceptable carrier. Pharmaceutical compositions of the current invention may further contain a biocide, antimicrobial, or antibiotic.

The term “subject” includes organisms which can suffer from infection. The term subject includes mammals, e.g., horses, monkeys, bears, dogs, cats, mice, rabbits, cattle, squirrels, rats, and, preferably, humans.

Molecules of the Invention

The present invention provides molecules, e.g., molecules comprising a reconstituted antibody mimetic. The molecules may further include a heterologous polypeptide or nonpeptidic molecule, e.g., a toxin.

Reconstituted antibody mimetics of the invention are designed based on antibodies that bind to a specific target molecule, cell or organism. For example, antibodies that are used to design the reconstituted antibody mimetics of the invention can be specific for a cell, e.g., a cancer cell, or for a bacteria, e.g., a pathogenic bacteria.

Reconstituted antibodies are useful in the methods of the invention. Reconstituted antibodies are of limited size but retain the ability to specifically recognize a target.

Reconstituted antibody mimetics of the invention are designed based on the sequence of known antibodies, e.g., monoclonal antibodies, polyclonal antibodies,

ScFv molecules that specifically recognize an antigen on the cell surface of a organism, e.g., a viral antigen, can be used as the organism targeting agent. Specifically, ScFV molecules that recognize viral particles, e.g., herpes virus particles, can be fused to a channel forming polypeptide or a channel forming domain thereof. Exemplary, non limiting ScFV molecules that are contemplated for use in the methods and compositions of the invention are HBV PreS1 ScFV, and HBV HBsAg ScFV.

Nucleic Acid Molecules of the Invention

Another aspect of the invention pertains to vectors, preferably expression vectors, containing a nucleic acid molecule encoding a polypeptides of the invention (or a portion thereof). As used herein, the term “vector” refers to a nucleic acid molecule capable of transporting another nucleic acid molecule to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid molecule of the invention in a form suitable for expression of the nucleic acid molecule in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., polypeptides comprising a organism targeting domain and a channel forming polypeptide).

The recombinant expression vectors of the invention can be designed for expression of the polypeptides of the invention in prokaryotic or eukaryotic cells. For example, the polypeptides can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerivisae include pYepSec1 (Baldari, et al., (1987) EMBO J 6:229-234), pMFa (Kudjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, the polypeptides can be expressed in insect cells using baculovirus expression vectors. Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J 8:729-733) and immunoglobulins (Banedji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

Another aspect of the invention pertains to host cells into which a nucleic acid molecule encoding a polypeptide of the invention is introduced within a recombinant expression vector or a nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a polypeptide of the invention can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding the polypeptide of the invention or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) the polypeptides of the invention. Accordingly, the invention further provides methods for producing polypeptides using the host cells of the invention. In one embodiment, the method comprises culturing the host cell of the invention (into which a recombinant expression vector encoding a polypeptide of the invention has been introduced) in a suitable medium such that a polypeptides of the invention is produced. In another embodiment, the method further comprises isolating the polypeptide from the medium or the host cell.

The host cells of the invention can also be used to produce non-human transgenic animals. For example, in one embodiment, a host cell of the invention is a fertilized oocyte or an embryonic stem cell into which coding sequences have been introduced. Such host cells can then be used to create non-human transgenic animals in which exogenous sequences have been introduced into their genome or homologous recombinant animals in which endogenous sequences have been altered. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, in which an endogenous gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a nucleic acid into the male pronuclei of a fertilized oocyte, e.g., by microinjection, retroviral infection, and allowing the oocyte to develop in a pseudopregnant female foster animal. The cDNA sequence encoding a polypeptide of the invention can be introduced as a transgene into the genome of a non-human animal. Intronic sequences and polyadenylation signals can also be included in the transgene to increase the efficiency of expression of the transgene. A tissue-specific regulatory sequence(s) can be operably linked to a transgene to direct expression of a protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a transgene in its genome and/or expression of mRNA in tissues or cells of the animals. A transgenic founder animal can then be used to breed additional animals carrying the transgene. Moreover, transgenic animals carrying a transgene encoding a protein can further be bred to other transgenic animals carrying other transgenes.

In another embodiment, transgenic non-humans animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

Methods of Making the Molecules of the Invention

As described above, molecules of the invention may be made recombinantly using the nucleic acid molecules, vectors, and host cells described above.

Alternatively, the organism targeting moiety can be made synthetically, or isolated from a natural source and linked to the channel forming moiety using methods and techniques well known to one of skill in the art.

Further, to increase the stability or half life of the compounds of the invention, the peptides may be made, e.g., synthetically or recombinantly, to include one or more peptide analogs or mimmetics. Exemplary peptides can be synthesized to include D-isomers of the naturally occurring amino acid residues to increase the half life of the molecule when administered to a subject.

Pharmaceutical Compositions

The antimicrobial molecules of the invention (also referred to herein as “active compounds”) of the invention can be incorporated into pharmaceutical compositions suitable for administration. Such compositions typically comprise the active compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

Methods of Treatment

The present invention provides therapeutic methods of treating a subject having an infection, e.g., a bacterial, viral, or fungal infection.

As used herein the term “infection” includes the presence of a microbe in or on a subject which, if its growth and/or virulence were inhibited, would result in a benefit to the subject. As such, the term “infection” in addition to referring to the presence of pathogens also includes normal flora which is not desirable, e.g., on the skin of a burn patient or in the gastrointestinal tract of an immunocompromised patient. As used herein, the term “treating” refers to the administration of a compound to a subject, for prophylactic and/or therapeutic purposes. The term “administration” includes delivery to a subject, e.g., by any appropriate method which serves to deliver the drug to the site of the infection. Administration of the drug can be, e.g., oral, intravenous, or topical (as described in further detail below).

In a one embodiment, a subject having an infection is treated with an effective amount of a compound of the invention such that the infection is treated. In a specific embodiment, the subject that is being treated is infected by an organism which is resistant to at least one antibiotic.

The compounds of the invention comprise organism targeting agents. As is seen in the examples, although the targeting agent is derived from on species, there is cross reactivity with other organisms. A skilled artisan would be able to test compounds of the invention for their effects on different organisms using routine experimentation.

In one embodiment, a composition of the invention is administered to a subject in combination with additional agents, e.g., other compositions of the invention or an antibiotic.

The composition of the invention can be administered to a subject in need of treatment in an effective amount using the pharmaceutical compositions described herein.

This invention is further illustrated by the following examples which should not be construed as limiting. The contents of all references, patents and published patent applications cited throughout this application are incorporated herein by reference.

### Exemplification

### EXAMPLE 1

### Staphylococcus Targeted Pheromonicin

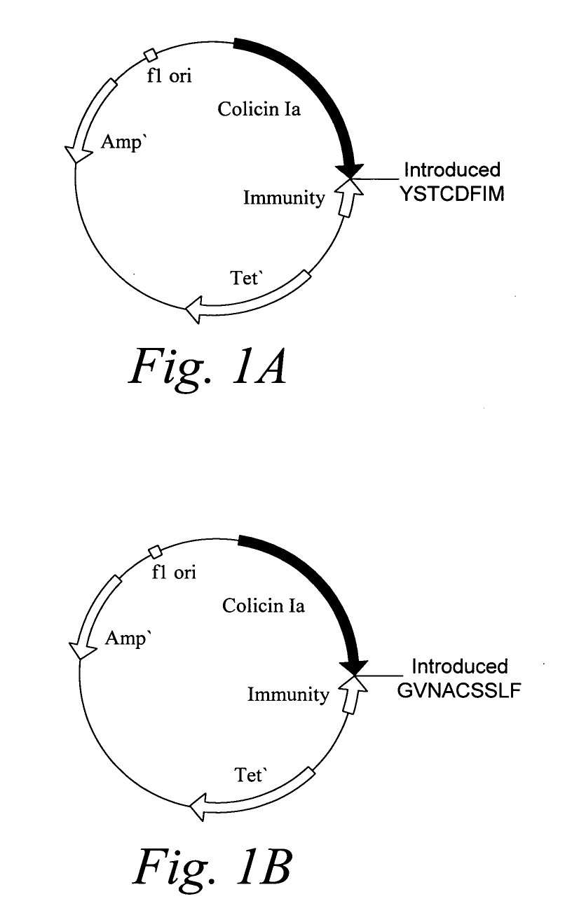

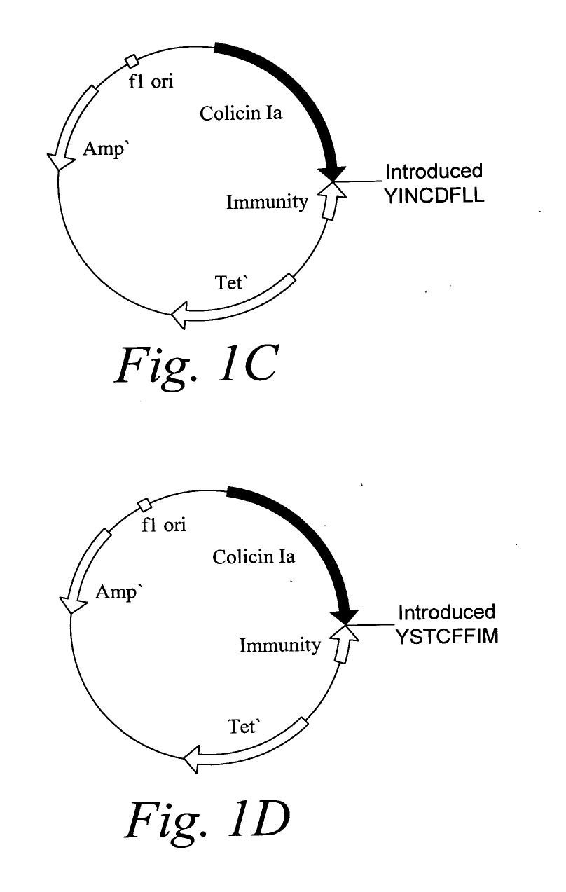

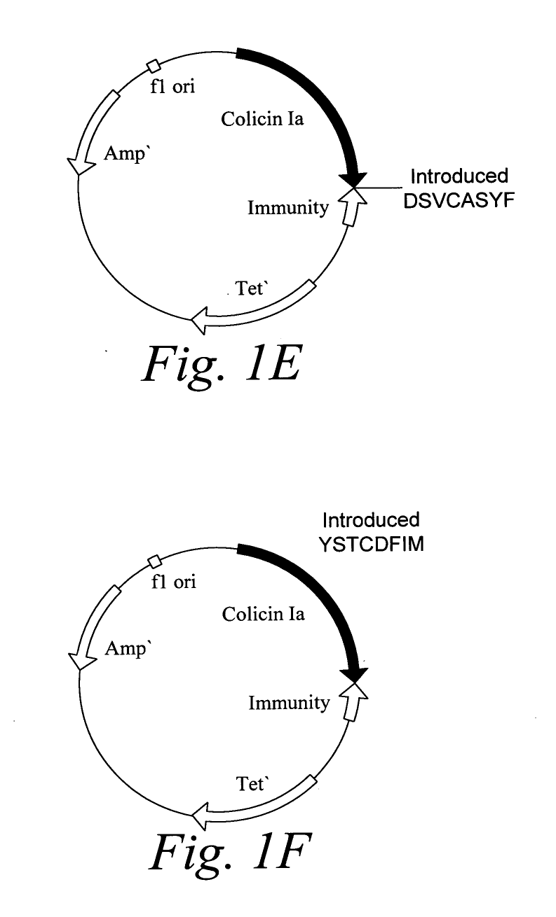

A Staphylococcal pheromone gene was inserted into positions I626 or S1 of colicin Ia gene located in pSELECT™-1 (Promega Co.) using the QuickChange™ Kit (Strategene Co.) (FIGS. 1A-F) (The polyeptide sequence of Colicin Ia is set forth as SEQ ID NO:30, and the nucleic acid molecule encoding Colicin Ia is set forth as SEQ ID NO:31). The mutagenesis was preformed according to the QuickChange directions and the plasmids were transfected into TG1 E. coli cells. TG1 cells harboring mutant plasmid were grown in FB medium to produce peptide. Peptides comprising a pheromone and a colicin, or channel forming domain thereof are referred herein as pheromonicin and abbreviated “Ph.”

In vitro Ph-SA Growth Inhibitory Activity

Penicillin-sensitive S. Aureus strain ATCC 25923 was used to investigate the effect of S. aureus ArgDI linked to colicin (Ph-SA) in vitro. 5 ul of ATCC 25923 cells (10⁵ CFU/ml) was inoculated into 10 ml liquid medium containing 1% tryptone, 1% NaCl, 0.5% yeast, 0.5% glucose and 1% HK₂PO₄, and were grown at 37° C. while shaking at 225 rpm.

Ph-SA1 (100 ng/ml), penicillin (PNC) (100 ng/ml), wild type colicin Ia (COL) (100 ng/ml), TG1 proteins from non-transfected TG1 cell (100 ng/ml), a control peptide wherein the C-terminus of colicin Ia was linked with other octapeptides (100 ng/ml) or a control (0.3M NaCI+50M borate buffer in the same amount as that of agents used) was added to each cell culture. The optical density of each culture was measured (A595 nm) every hr.

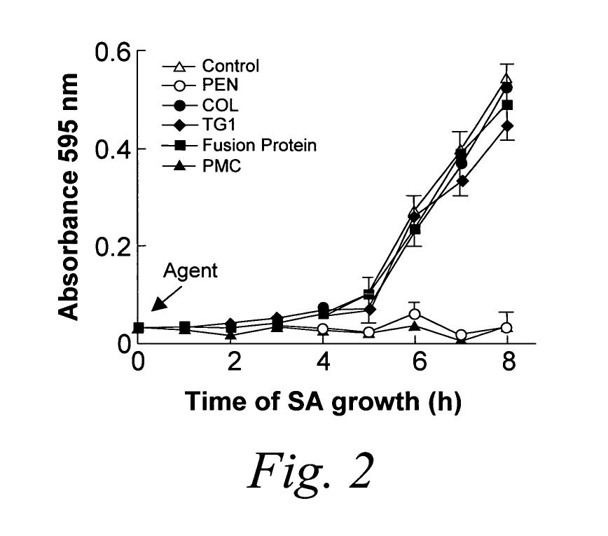

The results showed that both Ph-SA1 and penicillin inhibited more than 90% of cell growth but other agents failed (FIG. 2).

Electron Micrographs of Bactericidal Effects of Ph-SA1 Against ATCC BAA-42 MRSA Strain Cells (1% Phosphotungstic Acid. 25,000×)

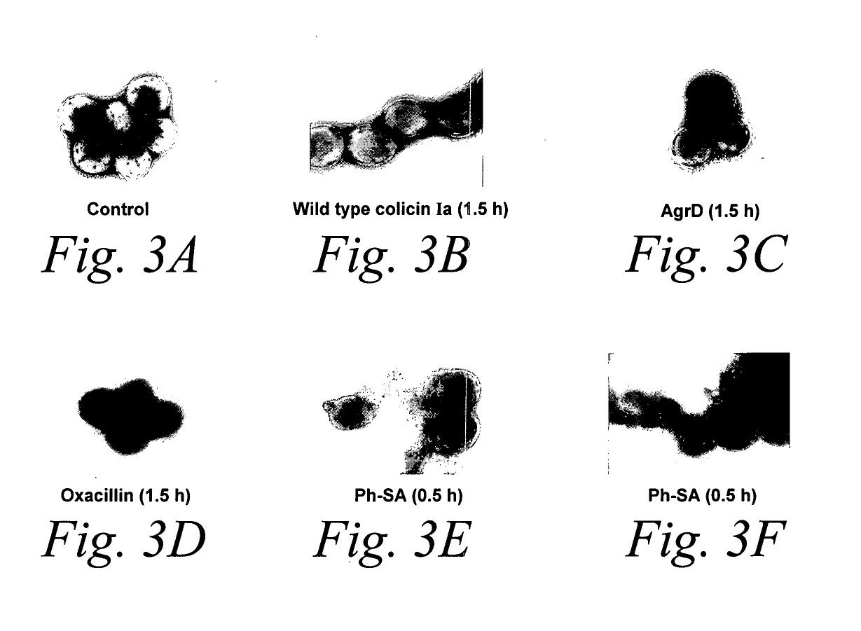

MRSA strain ATCC BAA-42 was treated with (a) nothing (control); (b) wild type colicin Ia (50 μg/ml) 2 hrs; (c) AgrDI (10 μg/ml) for 2 hrs; (d) oxacillin (500 μg/ml) for 2 hrs; or (e) and (f) Ph-SA (50 μg/ml) for 0.5 hrs. Cells were observed under a microscope at 25,000× (FIG. 3).

The results indicate that Ph-SA1 killed BAA-42 MRSA cells. Further, it appears that Ph-SA killed BAA-42 cells via a different mechanism than oxacillin. Free AgrD 1 and wild-type colicin Ia treatment had no effect on cell growth.

In vitro Ph-Stapyhylococci Growth Inhibitory Activity

The following experiments were performed by Lab. of Pharmacology, National Sichuan antibiotic industril institute, Chengdu, Sichuan.

The following agents were used to test the in vitro activity of Ph-SA:

(1) Ph-SA1 at 1.5 mg/ml, (2) Injection sodium Cephazoid, 0.5 g/bottle, (3) Injection sodium penicillin, 800,000 IU/bottle, (4) Injection sodium ampicillin, 1 g/bottle, and (5) Injection oxacillin, 0.5 g/bottle, (6) Vancomycin, 2.5 mg/ml. These agents were dissolved and diluted with sterilized water to the final concentrations of: 128 mg/L, 64 mg/L, 32 mg/L, 16 mg/L, L8 mg/L, L4 mg/L, 2 mg/L, 1 mg/L, 0.5 mg/L, 0.25 mg/L, 0.125 mg/L, 0.06 mg/L, 0.03 mg/L, and 0.015 mg/L,

All the bacterial strains used in the experiments were clinical isolates collected from Sichuan and Beijing in 2002 and identified by West China Hospital, Sichuan University and Chongqing University of Medical Sciences with API system. The following strains were used in the experiments detailed below: Staphylococcus aureus 168 strains (MRSA 80 strains and MSSA 88 strains) Staphylococcus epidermidis 46 strains (MRSE 25 strains and MSSE 21 strains), Enterococcus 20 strains, and Acenetobactor 20 strains. Staphylococcus aureus ATCC25923, 29213 and BAA-42 were used as control strains. Bacterial strains were contacted with one of the above described compounds and MIC50, MIC90 and MICScope were determined. The results are depicted in Table 2.

Results

TABLE 2
Comparision of the vitro antibacterial activity of Ph-SA1

| Strain | medicament | MIC50 | MIC90 | MIC Scope |
|---|---|---|---|---|
| (No. of strain) | mg/l | mg/l | mg/l | mg/l |
| Staphylococcus aureus | Ph-SA1 | 16> | 16 | <0.0005˜64 |
| MRSA strain | Cephazoid | >128 | >128 | 1˜128 |
| (80) | Penicillin | 128 | >128 | 1˜128 |
| | Ampicillin | 16 | 32 | 0.06˜64 |
| | Oxacillin | >128 | >128 | 0.5˜128 |
| | Vancomycin | 0.25 | 1 | 0.004˜2 |
| Staphylococcus aureus | Ph-SA1 | 0.5 | 16 | 0.125˜>64 |
| MSSA strain | Cephazoid | 0.125 | 0.25 | <0.015˜0.5 |
| (88) | penicillin | 8 | 64 | 0.125˜>128 |
| | Ampicillin | 0.25 | 1 | <0.015˜0.25 |
| | Oxacillin | 0.125 | 0.125 | <0.015˜0.25 |
| | Vancomycin | 0.5 | 1 | 0.03˜1 |
| Staphylococcus epidermidis | Ph-SA1 | >64 | >64 | 0.25˜>64 |
| MRSE strain | Cephazoid | 32 | >128 | 0.06˜>128 |
| (25) | penicillin | >128 | >128 | 8˜>128 |
| | Ampicillin | 16 | 64 | 0.06˜64 |
| | Oxacillin | 64 | >128 | 8˜>128 |
| | Vancomycin | 0.5 | 1 | 0.25˜2 |
| Staphylococcus epidermidis | Ph-SA1 | 0.25 | 16 | 0.125˜32 |
| MSSE strain | Cephazoid | 0.03 | 0.5 | <0.015˜0.5 |
| (21) | penicillin | 4 | 32 | 0.125˜128 |
| | Ampicillin | 0.03 | 0.25 | <0.015˜0.25 |
| | Oxacillin | 0.03 | 0.25 | <0.015˜0.25 |
| | Vancomycin | 0.5 | 0.5 | 0.06˜0.5 |
| Enterococcus | Ph-SA1 | >64 | >64 | 8˜>64 |
| (20) | Cephazoid | 32 | >128 | 32˜>128 |
| | penicillin | 32 | 64 | 32˜>128 |
| | Ampicillin | 0.5 | 16 | 0.25˜16 |
| | Oxacillin | 16 | >128 | 8˜>128 |
| | Vancomycin | 1 | 2 | 0.5˜50 |
| Acenetobactor | Ph-SA1 | 32 | >64 | 0.5˜>64 |
| (20) | Cephazoid | >128 | >128 | >128 |
| | Penicillin | >128 | >128 | >128 |
| | Ampicillin | >128 | >128 | 16˜>128 |
| | Oxacillin | >128 | >128 | >128 |
| | Vancomycin | >128 | >128 | 128˜>128 |

In vivo Ph-Staphylococci Protection Effects

The following experiments were performed by Pharmacology Lab, National Sichuan antibiotic industril institute, Chengdu, Sichuan.

The agents described above were diluted with sterilized water to final concentrations of 10 mg/L, 5 mg/Land 2.5 mg/L.

Staphylococcus aureus ATCC BAA-42(MRSA) and enterococcus faecalis ATCC 700802 were used as control strains.

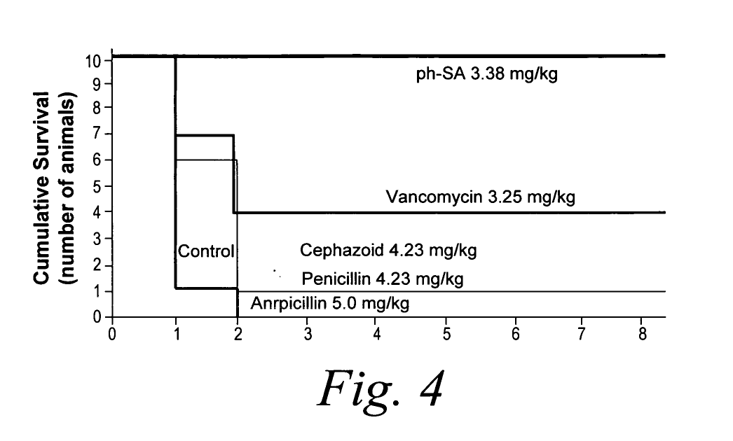

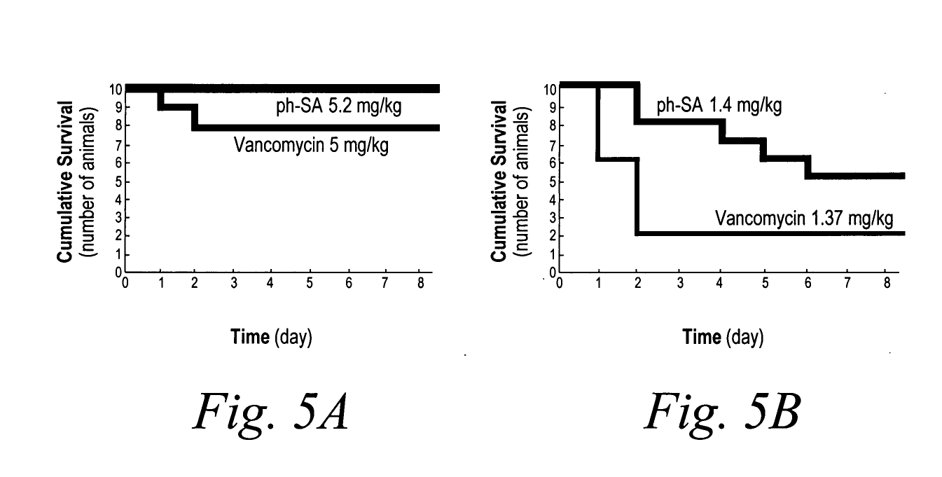

170 mice (Kungming mice: 18˜22 g, 85 males and 85 females) were randomly divided into four groups and injected with a fatal dosage of staphylococci, enterococcus and E. coli intraperitoneally. After one hour, mice were divided into control (n=10) and test groups (n=5) and received Ph-SA1, penicillin, ampicillin, cephazoid and vancomycin intravenously at 10 mg/kg, 5 mg/kg and 2.5 mg/kg, respectively. Mice were inspected every 24 hours for 7-14 days. Dead mice were counted as positive results. The results are depicted in Table 3 and FIGS. 4 and 5A-B.

TABLE 3
Ph-SA1's in vivo Protection

| | | | | | 95% of Confidence | |
|---|---|---|---|---|---|---|
| Bacteria& | | MIC | ED₅₀ | | Limit(mg/kg) | |
| (cfu/ml) | Medicament | (μg/ml) | mg/kg | | 1^{st} Time | 2^{nd} Time |
| Enterococcus | Ph-SA1 | 4 | 4.23 | (3.70) | 2.90-7.62 | (2.78-5.09) |
| faecalis | Cephazoid | >128 | >10 | (12.91) | >10 | (10.07-22.88) |
| ATCC 700802 | Penicillin | 2 | 3.16 | (3.08) | 2.37-5.06 | (2.38-3.91) |
| (8.0 × 10⁶) | Ampicillin | 0.125 | 3.37 | (3.00) | 2.51-5.77 | (1.65-4.71) |
| | vancomycin | 16 | 3.17 | (2.64) | 2.54-4.14 | (2.10-3.39) |
| Staphylococcus | Ph-SA1 | 0.5 | 4.32 | (4.23) | 3.28-6.18 | (3.05-5.86) |
| aureus | Cephazoid | 0.5 | 9.04 | (8.78) | 7.04-17.54 | (6.84-12.16) |
| MRSA BAA- | Penicillin | 8 | >10 | (14.71) | >10 | (11.29-21.19) |
| 42 | Ampicillin | 0.25 | 25.21 | (23.43) | 19.83-46.70 | (16.87-36.58) |
| (5.5 × 10⁵) | Vancomycin | 0.06 | 3.64 | (3.53) | 2.75-6.16 | (2.66-4.73) |
| | (0.08) | | | | | |
| Staphylococcus | Ph-SA1 | 0.5 | 3.84 | (4.24) | 2.65-5.32 | (2.54-7.08) |
| aureus | Cephazoid | 128 | 11.82 | (>10) | 8.55-25.02 | (>10) |
| MRSA 02-18 | Penicillin | >16 | 7.28 | (11.33) | 5.51-12.31 | (7.33-109.96) |
| (1.7 × 10⁵) | Ampicillin | 8 | 32.70 | (>20) | 25.51-53.88 | (>20) |
| | Vancomycin | 0.06 | 2.27 | (2.39) | 1.75-2.97 | (1.89-3.07) |
| | (0.077) | | | | | |
| Staphylococcus | Ph-SA1 | 0.25 | 10.35 | (8.87) | 8.04-15.38 | (7.00-11.85) |
| aureus | Cephazoid | 0.03 | 29.03 | (11.20) | 17.82-61.64 | (8.47-18.93) |
| ATCC 29213 | Penicillin | 16 | 10.75 | (9.84) | 8.42-15.79 | (7.68-14.04) |
| (3.6 × 10⁵) | Ampicillin | <0.015 | 14.25 | (12.26) | 10.00-44.51 | (9.80-18.29) |
| | Vancomycin | 0.5 | 2.32 | (2.09) | 1.71-3.29 | (1.57-2.76) |
| | (0.2) | | | | | |
| Escherichia | Ph-SA1 | 1 | 3.14 | (3.12) | 2.48-4.28 | (2.49-4.12) |
| coli | Cephazoid | >128 | >30.77 | (>30.77) | >30.77 | (>30.77) |
| 02-1-65 | | | | | | |
| (3.3 × 10⁵) | Penicillin | >128 | >30.77 | (>30.77) | >30.77 | (>30.77) |
| | Ampicillin | >128 | >30.77 | (>30.77) | >30.77 | (>30.77) |
| | vancomycin | 128 | >30.77 | (>30.77) | >30.77 | (>30.77) |

In vitro Ph-SE Growth Inhibition Results With S. epidermidis

The results indicated that Ph-SE inhibited the cell growth of regular S. epidermidis BQIOC 26069 from China Medical Bacteria Center, Beijing.

5 ul of cells (10⁵ CFU/ml) was inoculated into 10 ml liquid medium containing 1% tryptone, 1% NaCl, 0.5% yeast, 0.5% glucose and 1% HK₂PO₄, and grown at 37° C. while shaking at 225 rpm. The optical density of each culture was measured (A595 nm) every hour after treatment with control (Con), penicillin (PEN), Pheromonicin-S. aureus (Ph-SA1), or Pheromonicin-S. epidermidis (Ph-SE). All agents were at 2 ug/ml.

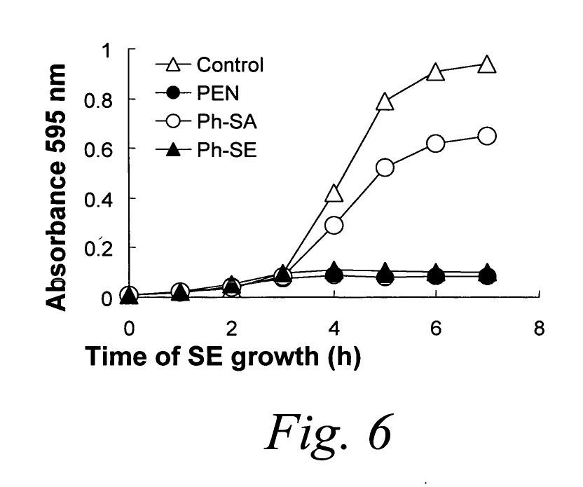

The results indicated that both Ph-SE and penicillin inhibited more than 90% of cell growth but Ph-SA1 only partially inhibited S. epidermidis cell growth (FIG. 6).

In vitro Pheromonicins Growth Inhibition Results

S. aureus strain ATCC BAA-42 MRSA was used. 5 ul of cells (10⁵ CFU/ml) was inoculated into 10 ml liquid medium containing 1% tryptone, 1% NaCl, 0.5% yeast, 0.5% glucose and 1% HK₂PO₄, grew in 37° C. with 225 rpm. The optical density value of medium was measured (A595 nm) every hour after treatment with Ph-SA1, Ph-SA2, Ph-SA3, Ph-SA4, Ph-SAR1 (ph-SA reverse), or oxacillin. All agents were 5 μg/ml.

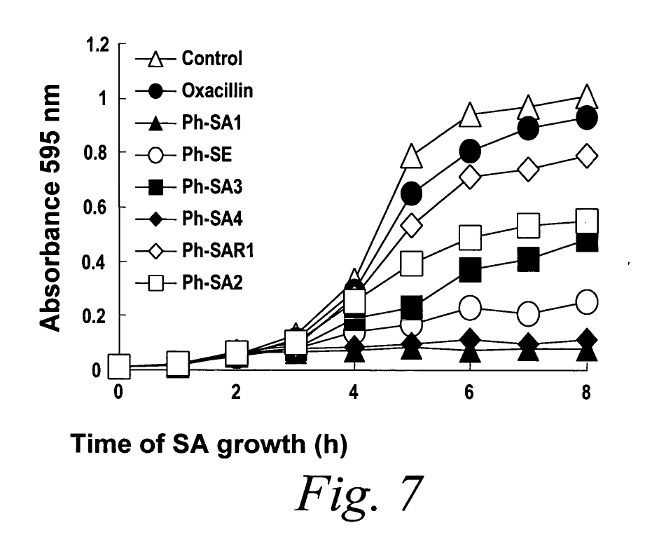

The results indicated that oxacillin and Ph-SAR1 did not inhibit the growth of ATCC BAA-42 MRSA. Ph-SA1 and Ph-SA4 had the best inhibitory effects on BAA-42 MRSA, Ph-SE had better effects, and Ph-SA2 and Ph-SA3 had some inhibition effects compared with Ph-SAR1 (FIG. 7).

### EXAMPLE 2

### Streptococcus Targeted Pheromonicin

Preparation of Ph-SP

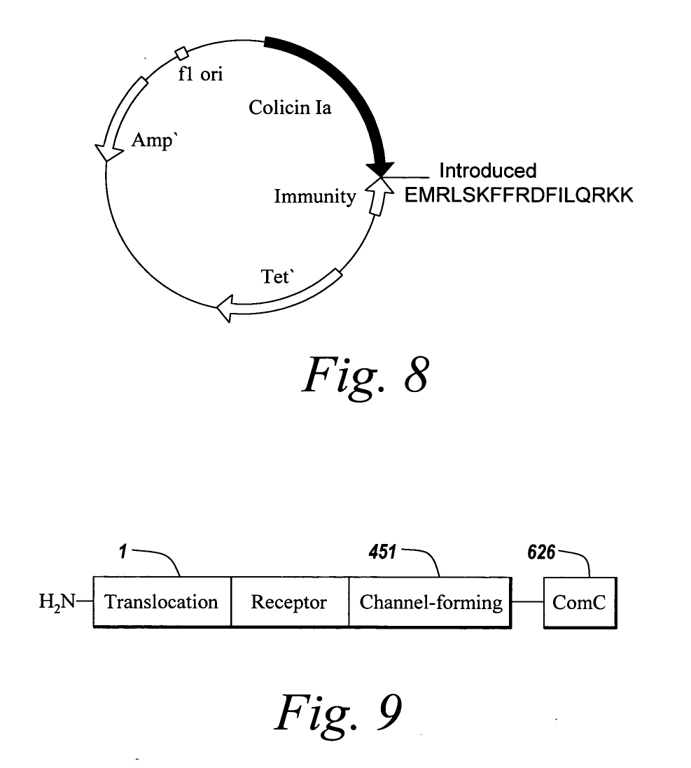

S. Pneumonia Com C pheromone gene was inserted in the position I626 of colicin Ia gene loaded in an 8.3 kb commercial plasmid, pSELECT™-1 (Promega Co.) using the QuickChange™ Mutagenesis Kit (Strategene Co.) (FIGS. 8 and 9). Mutagenesis was preformed according to the QuickChange protocol. Then mutant plasmid was transfected into the TG1 E. coli cells. TG1 cells harboring mutant plasmid were grown in FB medium to produce the Ph-SP peptide.

In vitro Growth Inhibitory Activity of Ph-SP

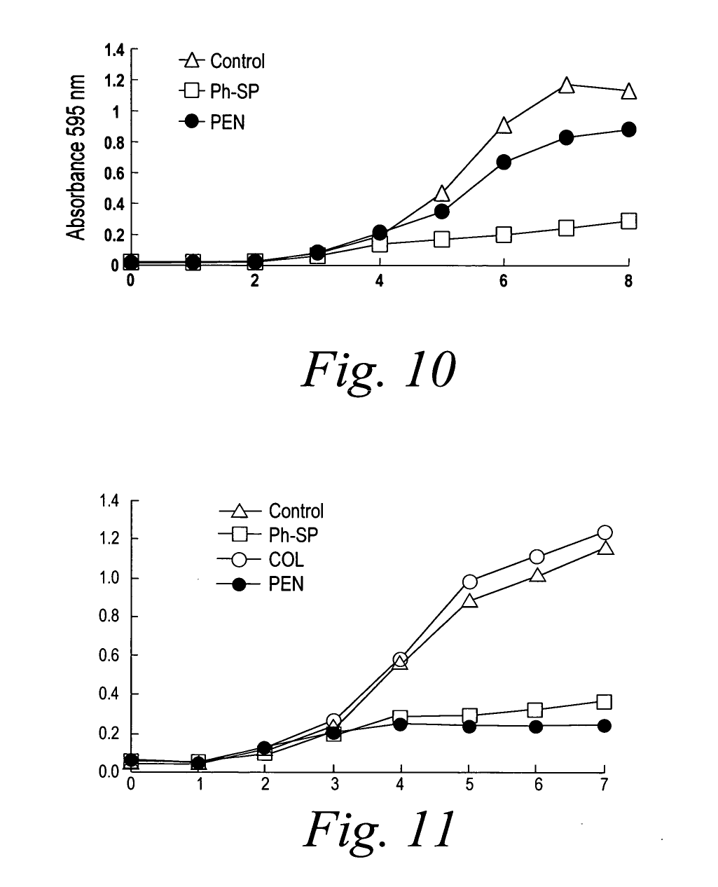

S. pneumoniae ATCC 700671 (Penicillin-resistant SP) and BQIOC 31201 (non-resistant S. pneumonia) from China Medical Bacteria Center, Beijing were used to test the activity of ph-SP in vitro. 5 ul of S. pneumoniae ATCC 700671 cells or BQIOC 31201 cells (10⁵ CFU/ml) was inoculated into 10 ml liquid medium containing 1% tryptone, 1% NaCl, 0.5% yeast, 0.5% glucose and 1% HK₂PO₄, and grown at 37° C. while shaking at 225 rpm. Ph-SP (5 ug/ml), penicillin (PNC) (5 ug/ml), wild type colicin Ia (COL) (5 ug/ml) and control (0.3M NaCl+50M borate buffer in the same amount as that of agents used) were added to each culture upon inoculation, respectively. The optical density of the cultures was measured (A595 nm) every hour. The results indicate that Ph-SP inhibited more than 90% of cell growth, while penicillin did not have a growth inhibitory effect on S. pneumoniae ATCC 700671(FIG. 10). The results further indicate that both Ph-SP and penicillin inhibited more than 90% of cell growth on S. pneumoniae BQIOC 31201, but wild type colicin Ia did not inhibit growth (FIG. 11).

Since the molecular weight of Ph-SP is 70,000 daltons and molecular weight of penicillin is 350 daltons, the bactericidal effect of Ph-SP was two hundred fold greater than that of penicillin on a molar basis.

To assess the in vivo effect of Ph-SP, mice were injected with ATCC 700671 PRSP intraperitoneally, followed by intravenous injections of PH-SP or controls.

Mice (20-25 g) were divided into four-groups: control group (n=10), penicillin group (n=15), cephalomycin group (n=15) and Ph-Sp group (n=15).

One hour after administration of the fatal dose of ATCC 700671 PRSP (10⁵ CFU/ml), each mouse was injected through the tail vein with test agents at three dosages as described in Table 4.

TABLE 4
In vivo protection effects of Ph-SP

| | | Number | | | | | | | | Number |
|---|---|---|---|---|---|---|---|---|---|---|
| | Dosage | of | Death distribution in hours | | | | | | | of |
| Agent | (mg/kg) | animal | 24 h | 48 h | 72 h | 96 h | 120 h | 144 h | 168 h | Death |
| Ph-SP | 10 | 5 | 0 | 0 | 0 | 0 | 0 | 0 | 0 | 0 |
| | 5 | 5 | 0 | 1 | 0 | 0 | 0 | 0 | 0 | 1 |
| | 2.5 | 5 | 0 | 0 | 1 | 1 | 0 | 0 | 0 | 2 |
| Penicillin | 10 | 5 | 3 | 0 | 0 | 0 | 0 | 0 | 0 | 3 |
| | 5 | 5 | 3 | 0 | 0 | 0 | 0 | 0 | 0 | 3 |
| | 2.5 | 5 | 5 | 0 | 0 | 0 | 0 | 0 | 0 | 5 |
| cephalomycin | 10 | 5 | 2 | 1 | 0 | 0 | 0 | 0 | 0 | 3 |
| | 5 | 5 | 3 | 1 | 0 | 0 | 0 | 0 | 0 | 4 |
| | 2.5 | 5 | 2 | 2 | 1 | 0 | 0 | 0 | 0 | 5 |
| Control | 0.5 ml | 10 | 9 | 1 | 0 | 0 | 0 | 0 | 0 | 10 |
| | 0.9% NaCl | | | | | | | | | |

The results indicate that Ph-Sp prevents streptococcus pneumoniae infection in mice.

### EXAMPLE 3

### Enterococcus Targeted Pheromonicin

Ph-EF Preparation

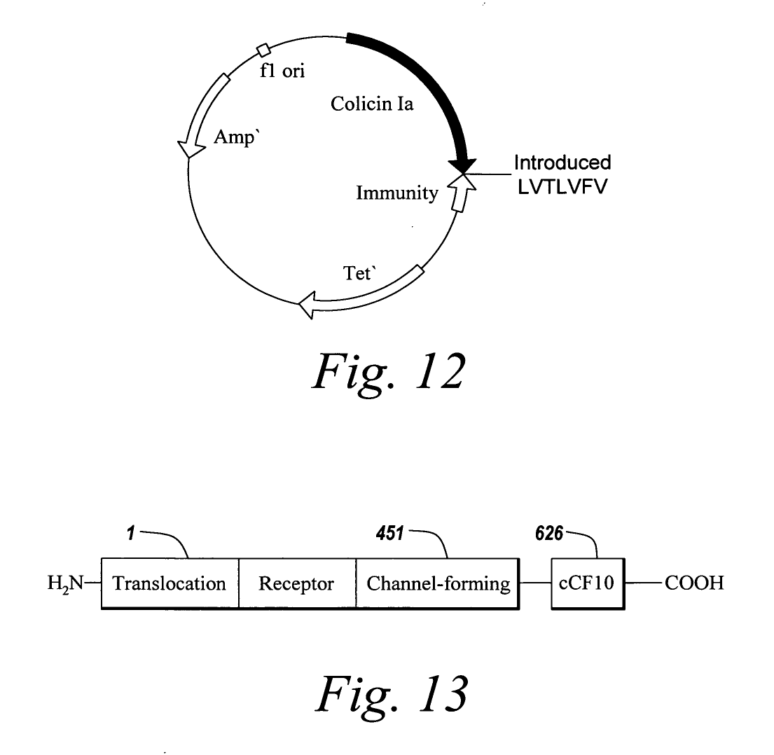

The gene encoding the Enterococcal cCF10 pheromone gene was inserted in to position I626 of colicin Ia gene located in an 8.3 kb commercial plasmid, pSELECT™-1 (Promega Co.) using the QuickChange™ Mutagenesis Kit (Strategene Inc.) (FIGS. 12 and 13). The mutant plasmid was transfected into the TG1 E. coli cells. TG1 cells harboring mutant plasmids were grown in FB medium to produce the pehromonicin peptide.

In vitro Growth Inhibitory Activity of Ph-EF

Vancomycin-resistant Enterococcus ATCC 700802and vancomycin-sensitive Enterococcus ATCC29212 were used to test the effects of enterococcus targeted pheromonicin in vitro. 5 ul of ATCC 29212 cells or ATCC 700802 cells (10⁵ CFU/ml) was cultured into 10 ml liquid medium containing 1% tryptone, 1% NaCl, 0.5% yeast, 0.5% glucose and 1% HK₂PO₄ at 37° C. while shaking at 225 rpm. Ph-EF, penicillin (PNC), vancomycin (VAN), wild type colicin Ia (COL) or control was added to each cultures, respectively. The optical density of each culture was measured (A595 nm) every hr.

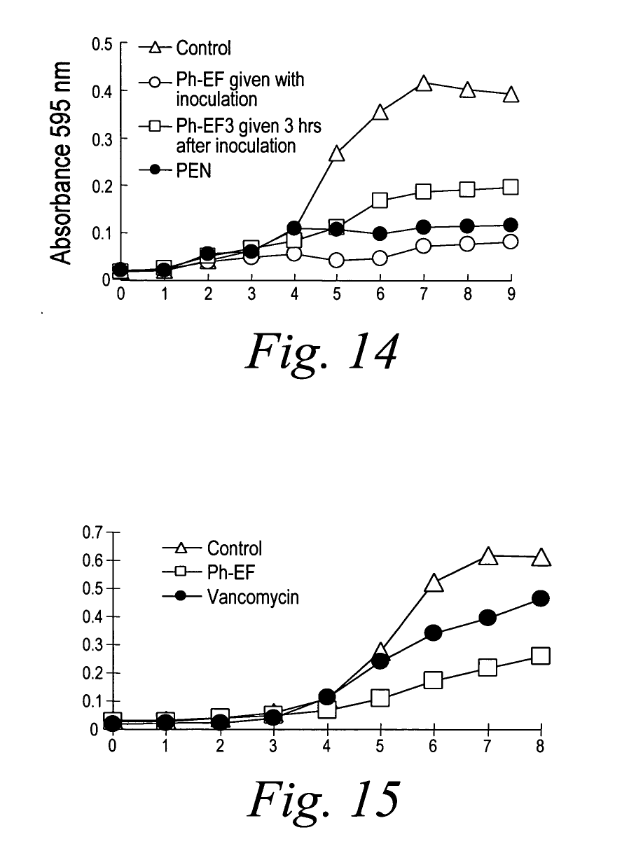

The results indicate that Ph-EF (3 ug/ml) inhibited cell growth as penicillin (3 ug/ml) did using ATCC 29212 cells, while wild type colicin Ia (3 ug/ml) failed to inhibit growth. The results further indicate that vancomycin (10 ug/ml) only partially inhibited VRE cell growth. In contrast, Ph-EF (10 ug/ml) effectively inhibited VRE cell growth (FIGS. 14 and 15).

Since the molecular weight of Ph-EF is 70,000 daltons and the molecular weight of vancomycin is 1456 daltons, the bactericidal effect of Ph-EF was almost one hundred times greater than that of vancomycin on a molar basis.

Electron Microraphs of Bactericidal Effects of Ph-EF Against ATCC 700802 VRE Cells (1% Phosphotungstic Acid, 15.000×).

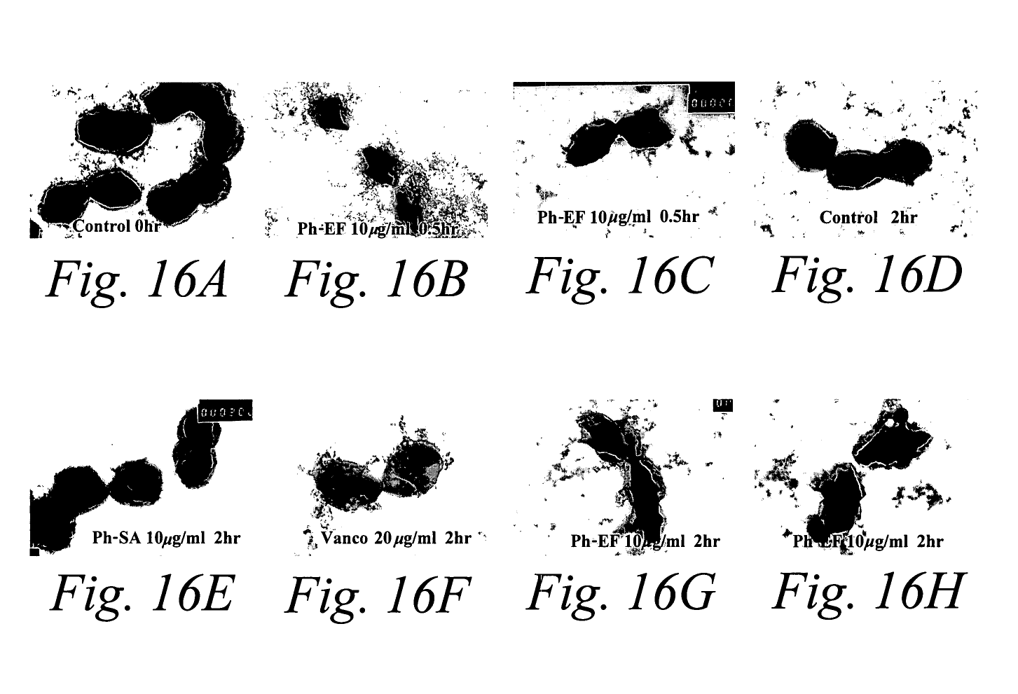

Bactericidal effects of ph-EF were tested and monitored by electron microscopy. FIG. 16 depicts electron micrographs of (a) untreated cells; (b) and (c) cells treated with Ph-EF (10 ug/ml) for 0.5 hr; (d) untreated cells for 2 hr; (e) cells treated by Ph-SA (10 ug/ml) for 2 hr; (f) cells treated with vancomycin (20 ug/ml) for 2 hr; (g) and (h) cells treated with Ph-EF (10 ug/ml) for 2 hrs.

The results indicated that Ph-SA and vancomycin treatment had no morphological alteration in VRE cells. In contrast, the Ph-EF treatment did result in substantial morphological alteration in VRE cells such as swelling cells and membrane damage.

Ph-EF Protection of ATCC 700802 Infected Mice

To assess in vivo effects of Ph-EF, mice were injected with a fatal dosage of ATCC 700802 VRE cells intraperitoneally. After one hour, mice were divided into control group (n=10) and treated subgroups (n=5) with Ph-EF, penicillin and vancomycin, given intravenously at 10 mg/kg, 5mg/kg and 2.5 mg/kg, respectively. Mice were inspected every 24 hours for 7-14 days. Dead mice would be counted as positive results. The results are depicted in Table 5.

TABLE 5
In vivo protection effects of Ph-EF

| | | | | | | | | | | Number |
|---|---|---|---|---|---|---|---|---|---|---|
| | Dosage | # of | Death distribution in hours | | | | | | | of |
| Agent | (mg/kg) | animal | 24 h | 48 h | 72 h | 96 h | 120 h | 144 h | 168 h | Death |
| Ph-EF | 10 | 5 | 0 | 0 | 0 | 0 | 0 | 0 | 0 | 0 |
| | 5 | 5 | 0 | 1 | 0 | 0 | 0 | 0 | 0 | 1 |
| | 2.5 | 5 | 0 | 2 | 1 | 1 | 0 | 0 | 0 | 2 |
| Penicillin | 10 | 5 | 0 | 0 | 0 | 0 | 0 | 0 | 0 | 0 |
| | 5 | 5 | 0 | 1 | 1 | 1 | 0 | 0 | 0 | 3 |
| | 2.5 | 5 | 1 | 2 | 1 | 0 | 0 | 0 | 0 | 4 |
| Vancomycin | 10 | 5 | 1 | 1 | 0 | 0 | 0 | 0 | 0 | 1 |
| | 5 | 5 | 3 | 2 | 1 | 0 | 0 | 0 | 0 | 3 |
| | 2.5 | 5 | 1 | 2 | 1 | 1 | 0 | 0 | 0 | 5 |
| Control | 0.5 ml | 10 | 3 | 3 | 1 | 0 | 3 | 0 | 0 | 10 |
| | 0.9% NaCl | | | | | | | | | |

The results indicated that Ph-EF effectively prevent Enterococcus infection in mice.

### EXAMPLE 4

### Viral Targeted Fusion Peptides

Preparation of HBV Antibiotic Peptides

Nucleic acid molecules encoding the N-terminus of a reconstituted mimetics HBsAg ScFV (GeneBank AF236816) peptide was linked with the C-terminus of colicin Ia, or its channel-forming domain, to form an antiviral peptide. Due to the changing lengths of peptide chains of a reconstituted mimetics of HBsAg ScFV and colicin Ia, the molecular weight of that antiviral peptide varies from about 10,000 Da to about 100,000 Da.

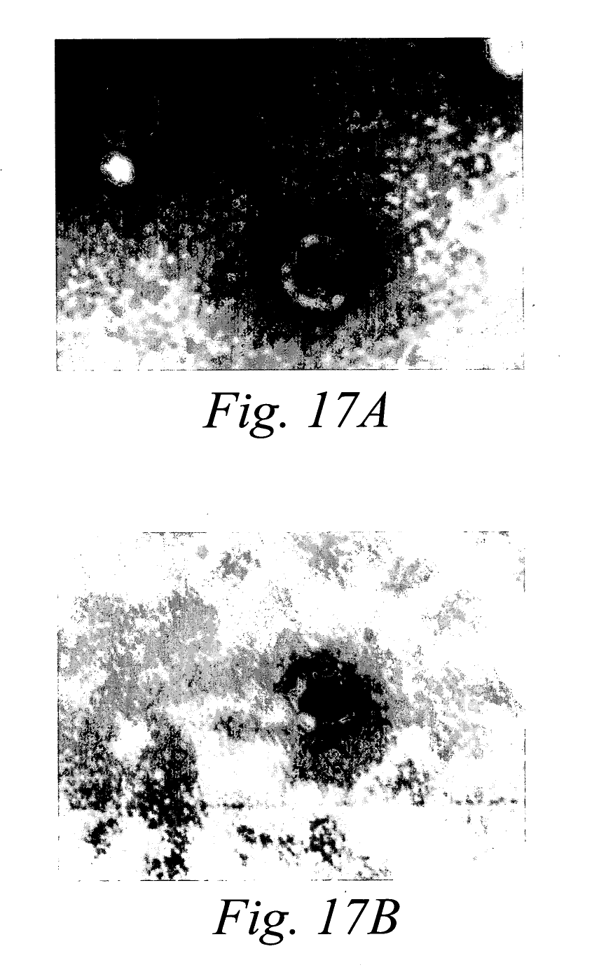

The genes encoding the polypeptide G28-V50/E217-S228 (heavy chain CDR1 linked to light chain CDR3 by the heavy chain FR2 peptide) of HBsAg scFV (GenBank AF236816) were inserted in to position I626 of the colicin Ia gene located in a 8.3 kb commercial vector, pSELECT™⁻¹ (Promega Co.) using the QuickChange™ Mutagenesis Kit (Strategene) (FIG. 17A). The mutant plasmid was transfected into the TG1 E. coli cells, and cells harboring the plasmid were grown in FB medium to produce the polypeptide. The polypeptide was purified by ion exchange chromatography.

The gene encoding HBV PreS1 scFV (GenBank accession No. AF427148) was linked at the C-terminus of a pore-forming region of Colicin Ia (amino aicd residues 544-626) to produce a recombinant polypeptide with a molecular weight of about 40,000 daltons (SEQ ID NO: 32). The nucleic acid molecule encoding SEQ ID NO: 32 is set forth as SEQ ID NO:33. To create a second polypeptide construct, the nucleic acid encoding a reconstituted mimetic of HBV PreS1 scFV (GenBank access No. AF427148) was linked to the C-terminus of Colicin Ia to create a polypeptide of molecular weight ˜90,000 (SEQ ID NO:34). The nucleic acid molecule encoding SEQ ID NO: 34 is set forth as SEQ ID NO:35.

In vitro Antiviral Effect of antiHBV Peptide on HBV

HBV (Dane's particle) was obtained from the centrifugation of culture fluid of HepG 2 2.15 cells (from Lab. of Hepatitis, Infectious Disease Division, West China Hospital, Sichuan University) at 100,000 g for 3 hr at 4° C. HBV was incubated with antiHBV peptide comprising the peptide of G28-V50/E217-S228 of HBsAg ScFV (Genbank AF236816) and colicin Ia, 10 ug/ml, for 1 hr, 37 ° C. Several micro litters of sample were stained by 1% phosphotungstic acid. EM observation indicated that viral envelope had been broken (FIG. 17).

In another set of experiments, HepG 2.2.15 cells transfected with HBV gene were treated with a peptide comprising the peptide of G28-V50/E217-S228 of HBsAg ScFV (Genbank AF236816) and colicin Ia (SEQ ID NO:36). The nucleic acid molecule encoding SEQ ID NO: 36 is set forth as SEQ ID NO:37. Cells were treated with 10 ug/ml of the peptide and allowed to grow in 1640 medium for 66 hours. Cells were observed under a microscope at 400×. Control cells displayed normal morphology and growth. Cells treated with the antiviral peptide showed altered morphology and necrosis as compared to the control cells.

Construction of Reconstructed Antibody-Colicin Antiviral Peptides and Effects on Hepatitis C Virus Infected Cells

SMMC-7721 human hepatocellular carcinoma cells (Institute of Biochemistry & Cell Biology, Shanghai, Chinese Academy of Science) were transfected with full HCV gene and grown with 1640 medium+10% fetal bovine serum. The concentration of antiviral peptides used for the treatment was about 100 micrograms per ml. Equal amounts stock solution (20 mM PBS, pH 7.4) was added to the control cells. The control cells and treated cells were observed under a microscope and the number of cells were counted, and their morphological features noted. Cell count and morphological features of the treatment groups were compared to the control groups.

SMMC-7721 cells transfected with HCV which were treated (as described above) with a peptide comprising the peptides of S31-G49/A219-H226 of HBV PreS-1 ScFV linked to Colicin Ia. Cell swelling occurred in certain cells in comparison with the control. The peptide comprised two CDRs from PreS-1 (Genbank AF427148) of HBV ScFv (as described above), linked to colicin Ia as shown below.

| | |
|---|---|
| NH₂-colicin Ia-I626-SYAIS- | (SEQ ID NO:38) |
| WVRQAAGQGLEWMG-AAWDDSLH-COOH | |

The nucleic acid molecule encoding SEQ ID NO:38 is set forth as SEQ ID NO:39.

SMMC-7721 cells transfected with HCV gene which were treated (as described above) with a peptide comprising the peptide from HBV PreS-1 ScFV linked to Colicin Ia. Cell swelling occurred in certain cells in comparison with the control. Specifically, the peptide comprised two CDRs from HBV PreS-1 ScFv (Genbank AF427148); CDR2 (Sequence: GIIPKFGTPNYIQKFQGR; SEQ. ID NO. 40) from the heavy chain variable region and a CDR2 portion (Sequence: YRNNQRPS; SEQ. ID NO.41) from the light chain variable region. The CDRs were linked by the natural CDR1-CDR2 light chain linker (Sequence: WYQQLPGTAPKLLI; SEQ. ID NO. 42), and the N-terminus of above peptide was linked to colicin Ia at residue position 626; the resultant peptide is illustrated below:

| | |
|---|---|
| NH₂-colicinIa-I626- | (SEQ ID NO:43) |
| GIIPKFGTPNYIQKFQGR-WYQQLPGTAPKLLI- | |
| YRNNQRPS-COOH | |

The nucleic acid molecule encoding SEQ ID NO:43 is set forth as SEQ ID NO:44.

SMMC-7721 cells transfected with HCV gene which were treated (as described above) with a peptide comprising the peptide of G28-V50/E217-S228 from HBV HBsAg ScFV linked to Colicin Ia. Most of the treated cells died in comparison with the control. The peptide comprised two segments of HBV HBsAg ScFv (Genbank AF236816); CDR1 (Sequence: GFTFSDYYMS; SEQ. ID NO:45) from the heavy chain variable region and CDR3 (Sequence: DEADYYCNSRDS; SEQ. ID NO:46) from the light chain variable region. The portions CDR1 and CDR3 were linked by the natural CDR1-CDR2 linker (Sequence: WIRQAPGKGLEWVS; SEQ. ID NO:47) from the heavy chain. The CDR fusion was linked to colicin Ia at residue position 626; the resultant peptide is illustrated below:

| | |
|---|---|
| NH₂-colicinIa-I626-GFTFSDYYMS- | (SEQ ID NO:48) |
| WIRQAPGKGLEWVS-DEADYYCNSRDS- | |
| COOH | |

The nucleic acid molecule encoding SEQ ID NO:48 is set forth as SEQ ID NO:49.

SMMC-7721 cells transfected with HCV gene which were treated (as described above) with a peptide comprising the peptide of G28-S37 from HBV HBsAg ScFV linked to Colicin Ia. Some of the treated cells died in comparison with the control and the rest became swollen and/or deformed. Specifically, the peptide comprised CDR1 (Sequence: GFTFSDYYMS (SEQ ID NO:50) of the heavy chain variable region HBV HBsAg ScFv (Genbank AF236816). CDR1 was linked to colicin Ia at residue position 626; the resultant peptide is illustrated below:

| | |
|---|---|
| NH₂-colicinIa-I626-GFTFSDYYMS-COOH | (SEQ ID NO:51) |

The nucleic acid molecule encoding SEQ ID NO:51 is set forth as SEQ ID NO:52.

SMMC-7721 cells transfected with HCV gene which were treated (as described above) with a peptide comprising the peptide of E217-S228 from HBV HBsAg ScFV linked to Colicin Ia a ScFv targeting the HBV HBsAg antigen. A portion of the treated cells died in comparison with the control and the rest became swollen and/or deformed. Specifically, the peptide comprised CDR3 (Sequence: DEADYYCNSRDS (SEQ ID NO:53)) from the light chain variable region of HBV HBsAg ScFv (Genbank AF236816). The CDR3 portion was linked to colicin Ia at residue position 626; the resultant peptide is illustrated below:

| | |
|---|---|
| NH₂-colicinIa-I626-DEADYYCNSRDS-COOH | (SEQ ID NO:54) |

The nucleic acid molecule encoding SEQ ID NO:54 is set forth as SEQ ID NO:55.

Toxicity Assessment

To assess potential toxicity to mammalian cells, HL-7702 human liver cells and SMMC-7721 human hepatocellular carcinoma cells (Institute of Biochemistry & Cell Biology, Shanghai, Chinese Academy of Science) were grown in 1640 medium and treated with various fusion peptides. The concentration of antivirus and antibacterial peptides used for treating the cells was about 200 micrograms per ml. Equal amounts of stock solution (20 mM PBS, pH 7.4) was added to the control cells. The control cells and treated cells were observed with a microscope at 400× magnification to count the number of cells and compare morphological alterations with respects to the control group.

It was observed that all reconstituted mimetics of PreS-1 and HBsAg ScFV-colicin Ia peptides of described above changed neither treated cell morphology nor treated cell growth in comparison with untreated cells after 48 hours incubation. It was further determined that treatment of HL-7702 human liver cells and SMMC-7721 human hepatocellular carcinoma cells with an antibiotic peptide, anti-Staphylococcus aureus peptide (Ph-SA peptide) was also not toxic to the liver cells.

Compositions for the Treatment of Chicken Newcastle Virus

Newcastle disease in chickens (also known as Ranikhet disease) is a serious and commonly fatal disease of chickens caused by a virus classified within the genus Paramyxovirus of the family Paramyxoviridae. Morbidity rates and fatality rates for chickens can be as high as 100% and 90% respectively. In most developing countries, Newcastle disease is the most important infectious disease affecting chickens.

Antiviral peptides specific for chicken new castle virus were constructed.

| | |
|---|---|
| NH2-colicin-TLTTKLY-COOH | (SEQ ID NO:56) |
| NH2-colicin-CTLTTKLYC-COOH | (SEQ ID NO:57) |

The nucleic acid molecules encoding SEQ ID NO:56 and 57 are set forth as SEQ ID NO:58 and 59, respectively.

The peptides (TLTTKLY and CTLTTKLYC) come from the flamentous bacteriophage M13 and are (see, e.g., “Novel peptides that inhibit the propagation of Newcastle disease virus”, Archives of Virology, 147:981-983,2002 by P.Ramanujam et al.; the entire contents of which is hereby incorporated by reference).

Colicin Ia linked with TLTTKLY or CTLTTKLYC peptides could bind with the envelope antigen of Chicken New Castle virus by competing with the antibody. In primary isolated chicken embryo leg-muscle cell culture, we found this peptide could kill the majority of infected chicken muscle cells. Several hours after the peptide was added, most of cells had died.

### EXAMPLE 5

### Fungal Targeted Fusion Peptides

A fusion peptide pheromonicin-CA (Ph-CA) was created by incorporating the peptide chain of colicin Ia with the Candida albicans alpha mating pheromone, GFRLTNFGYFEPG (SEQ ID NO:60). The oligonucleotides encoding the Candida albicans alpha mating pheromone was introduced at the C-terminus (I626) of colicin Ia gene to form a gene that encodes a 639-residue peptide.

A second fusion peptide, reverse pheromonicin-CA (Rev. Ph-CA), was created by incorporating the peptide chain of colicin Ia with a Candida albicans alpha mating pheromone, GFRLTNFGYFEPG by introducing the oligonucleotides encoding Candida albicans alpha mating pheromone at N-terminus (S1) of colcin Ia gene to form a gene that encodes a 639-residue peptide (SEQ ID NO:61). The nucleic acid molecule encoding SEQ ID NO:61 is set forth as SEQ ID NO:62.

In vivo and in vitro Effects of Ph-CA and Rev. Ph-CA

Ph-Ca had a pronounced antifungal effect against Candida albicans in vitro and in vivo. In contrast, Rev. Ph-CA had no effect.

Candida albicans ATCC 10231 was used to test the effects of Ph-CA and rev. Ph-CA. In vitro cell growth inhibition assays were performed in 100 ml Klett flasks containing 10 ml of MH medium and monitored turbimetrically with a BioRad 550 microplate reader at OD595 nm by every 60 min. The filaments (mycelium) precipitated to the bottom of the flask were monitored with a digital photo-recorder every 6 hrs. Cells were inoculated to an initial cell density ˜2.5×10⁵ CFU/ml and shaken at 200 rpm on an orbital shaker at 35° C.

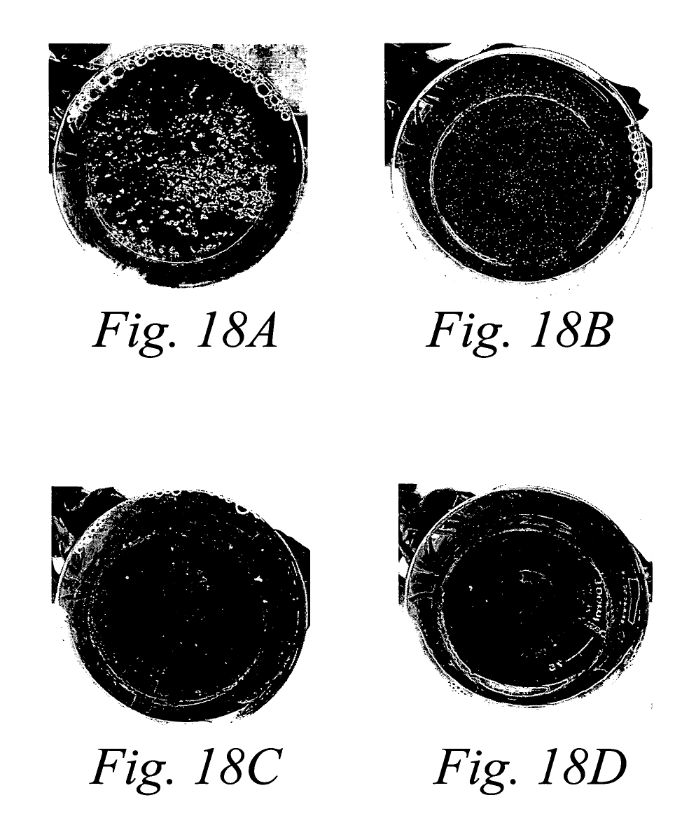

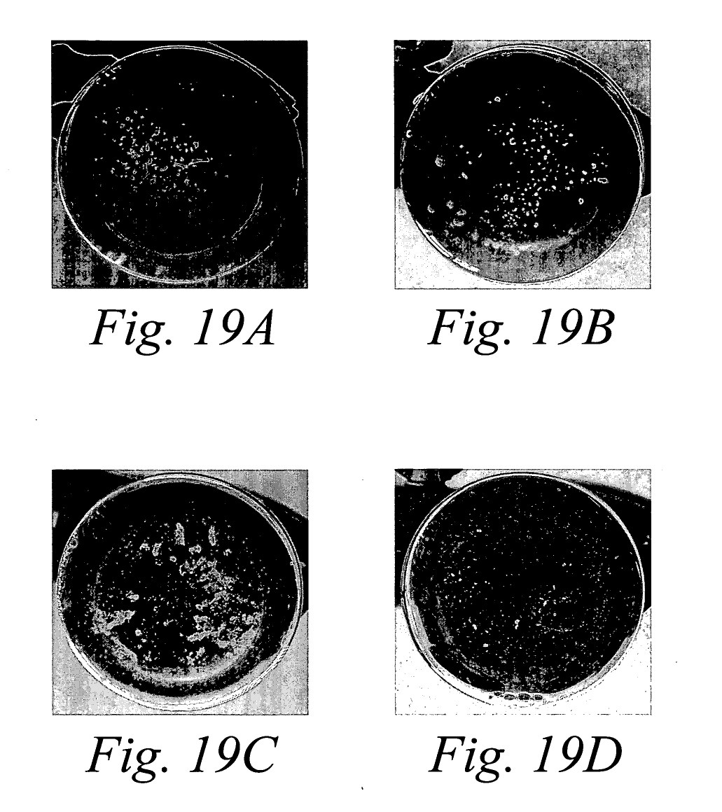

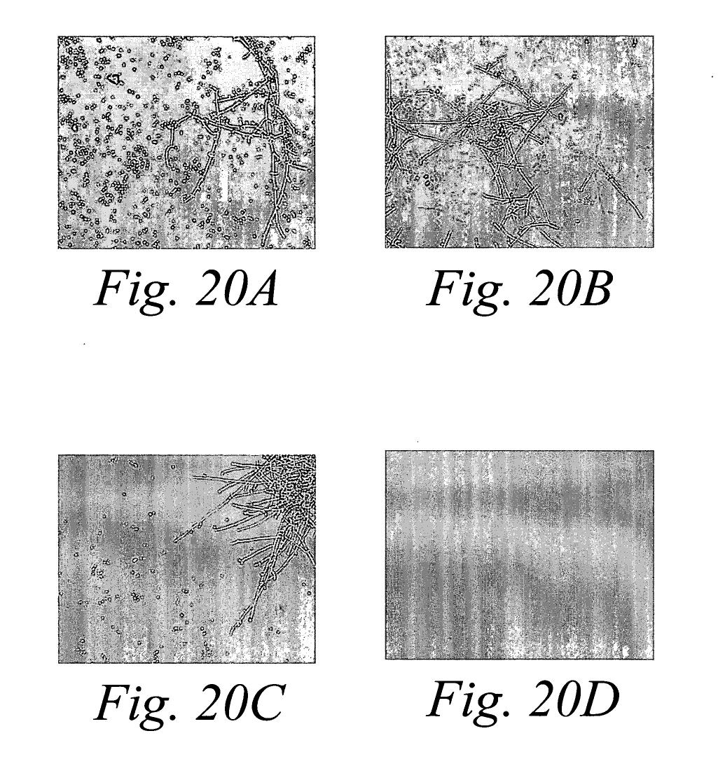

Ph-CA and Rev. Ph-CA were added upon inoculation of the culture. The same amount of borate stock solution (50 mM borate, pH 9.0), Ph-SA (pheromonicin constructed by colicin Ia and staphylococcal pheromone AgrD1) (5 μg/ml) and three antibiotics preparations (50 μg/ml ampicillin, 200 ng/ml amphotericin B, 3 μg/ml fluconazole) were used as controls. All assays were expressed in turbidometric absorbance units measured at 595 nm and documented by photocopying the filaments sedimented at the bottom of flask (see, FIGS. 18 and 19). Samples from the flasks were observed under a light microscope (FIG. 20).

Ampicillin, fluconazole, and Ph-SA had no effect on the growth of ATCC strain 10231 of Candida albicans compared to untreated controls. In contrast, 5 μg/ml Ph-CA completely inhibited C. albicans growth, as did 200 ng/ml amphotericin B.

20 μg/ml Rev. Ph-CA had about 30% of the inhibition effect as compared to PMC-CA. Considering the difference in molecular weight between Ph-CA (70 kDa) and amphotericin B (about 0.85 kDa), the inhibitory effect of Ph-CA against Candida albicans strain ATCC 10231 was approximately three times greater, on a molar basis.

To assess the potential toxicity to mammalian cells, ATCC CCL-13 Chang liver cell lines were incubated in medium with Ph-CA (200 μg /ml). No obvious differences in cell counts or lactate dehydrogenase levels in cultured cells were observed when compared to controls. These data suggested that unlike current antifungal drugs such as amphotericin B, Ph-CA is tolerated by mammalian systems without evident toxicity.

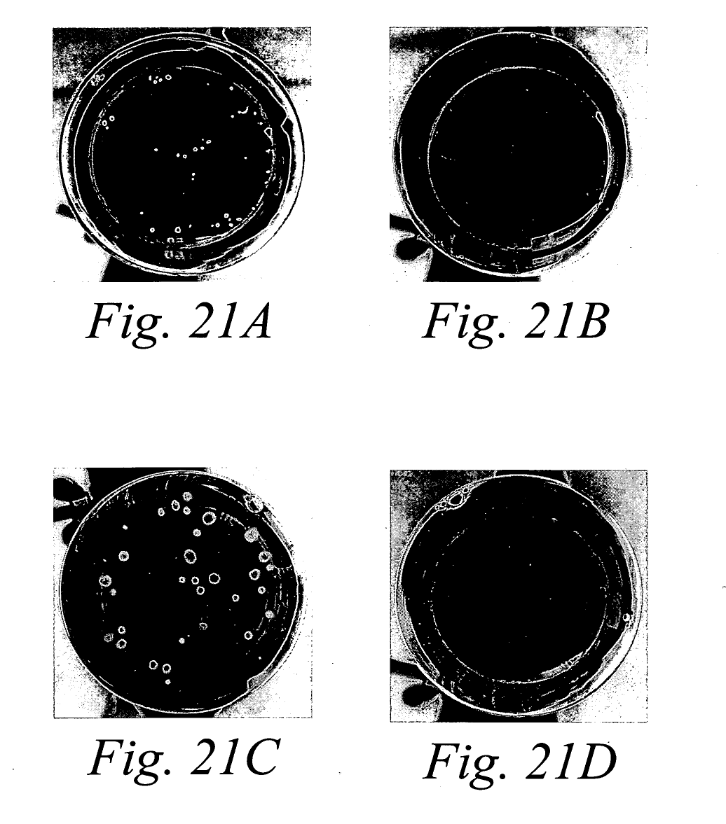

The same in vitro cell growth inhibition assays were performed on Aspergillus niger (SWA 1011 strain from Bio-technical Center, Southwestern Agriculture University, China). 5 μg/ml Ph-CA inhibited Aspergillus niger growth, as did 200 ng/ml amphotericin B. 3 μg/ml Fluconazole and 5 μg/ml Ph-SA had no effect on the growth of Aspergillus niger (FIG. 21).

As indicated by the results described herein, chimeric polypeptide such as those described above are valuable as antibiotics against fungal infections, e.g., Candida albicans and Asperigillis niger.

Equivalents

Those skilled in the art will recognize, or be able to ascertain using no more than routine experimentation, many equivalents to the specific embodiments of the invention described herein. Such equivalents are intended to be encompassed by the following claims.

1. A polypeptide comprising an organism targeting agent covalently attached to a channel forming moiety.

2. (canceled)

3. The polypeptide of claim 1, wherein said channel forming moiety is a channel forming polypeptide or a channel forming fragment thereof.

4. The polypeptide of claim 3, wherein said channel forming polypeptide, or channel forming fragment thereof, is selected from the group consisting of: α-hemolysin, diphtheria toxin, delta toxin, anthrax toxin, and colicin.

5. The polypeptide of claim 4, wherein said channel forming polypeptide, or channel forming fragment thereof, is colicin.

6. The polypeptide of claim 5, wherein said colicin is selected from the group consisting of E1, Ia, Ib, A, B and N.

7. The polypeptide of claim 6, wherein said colicin is colicin Ia.

8. The polypeptide of claim 7, wherein said channel forming fragment of colicin Ia is selected from the group consisting of amino acid residues 544-626 and 524-626:

9. The polypeptide of claim 1, wherein said organism targeting agent is selected from the group consisting of a ligand, an antibody, or fragment thereof, a phage segment, and a pheromone.

10. (canceled)

11. The polypeptide of claim 10 wherein said organism targeting agent is a pheromone.

12. (canceled)

13. (canceled)

14. (canceled)

15. (canceled)

16. (canceled)

17. (canceled)

18. (canceled)

19. (canceled)

20. (canceled)

21. (canceled)

22. The polypeptide of claim 1, wherein said organism targeting agent is C-terminal to said channel forming moiety.

23. The polypeptide of claim 1, wherein said organism targeting agent is N-terminal to said channel forming moiety.

24. The polypeptide of claim 10, wherein said organism targeting agent is an antibody or a reconstituted antibody mimetic.

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. A polypeptide comprising a fungal pheromone and a channel forming domain of colicin.

37. The polypeptide of claim 36, wherein said channel forming domain of colicin comprises residues 544-626 of colicin Ia.

38. The polypeptide of claim 1, comprising non-natural amino acid residues.

39. The polypeptide of claim 38, wherein said non-natural amino acid residues are amino acid analogs, or mimetics.

40. The polypeptide of claim 38, wherein said non-natural amino acid residues are D-isomers of natural amino acid residues.

41-72. (canceled)

73. The polypeptide of claim 36 wherein said fungal pheromone is the Candida Albicans alpha mating pheromone.


SEQUENCE LISTING (62 sequences)

SEQ ID NO: 1 - protein, 633 aa, Artificial sequence
  Colicin attached to Pheromone peptide encoding Staphylococcus aureus AgrDI
SDPVRITNPGAESLGYDSDGHEIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVAEWDKLRQRLLDARNKITSAESAVNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVRTENWRPLFVKTETIIAGNAATALVALVFSILTGSALGIIGYGLLMAVTGALIDESLVEKANKFWGIYSTCDFIM

SEQ ID NO: 2 - DNA, 1905 bp, Artificial sequence
  Colicin attached to Pheromone peptide encoding Staphylococcus aureus AgrDI
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatcattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatacgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtatttattccacctgtgattttataatgtaa

SEQ ID NO: 3 - protein, 634 aa, Artificial sequence
  Colicin attached to Pheromone peptide encoding Staphylococcus aureus AgrDII
SDPVRITNPGAESLGYDSDGHEIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVAEWDKLRQRLLDARNKITSAESAVNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVRTENWRPLFVKTETIIAGNAATALVALVFSILTGSALGIIGYGLLMAVTGALIDESLVEKANKFWGIGVNACSSLF

SEQ ID NO: 4 - DNA, 1908 bp, Artificial sequence
  Colicin attached to Pheromone peptide encoding Staphylococcus aureus AgrDII
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatcattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatacgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtattggagttaacgcatgttcttccctgttttaa

SEQ ID NO: 5 - protein, 633 aa, Artificial sequence
  Colicin attached to Pheromone peptide encoding Staphylococcus aureus AgrDIII
SDPVRITNPGAESLGYDSDGHEIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVAEWDKLRQRLLDARNKITSAESAVNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVRTENWRPLFVKTETIIAGNAATALVALVFSILTGSALGIIGYGLLMAVTGALIDESLVEKANKFWGIYINCDFLL

SEQ ID NO: 6 - DNA, 1905 bp, Artificial sequence
  Colicin attached to Pheromone peptide encoding Staphylococcus aureus AgrDIII
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatcattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatacgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtatttatataaactgtgattttcttctgtaa

SEQ ID NO: 7 - protein, 633 aa, Artificial sequence
  Colicin attached to Pheromone peptide encoding Staphylococcus aureus AgrDIV
SDPVRITNPGAESLGYDSDGHEIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVAEWDKLRQRLLDARNKITSAESAVNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVRTENWRPLFVKTETIIAGNAATALVALVFSILTGSALGIIGYGLLMAVTGALIDESLVEKANKFWGIYSTCYFIM

SEQ ID NO: 8 - DNA, 1905 bp, Artificial sequence
  Colicin attached to Pheromone peptide encoding Staphylococcus aureus AgrDIV
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatcattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatacgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtatttattccacctgttactttataatgtaa

SEQ ID NO: 9 - protein, 633 aa, Artificial sequence
  Colicin Ia attached to Pheromone peptide from Staphylococcus epidermidis
SDPVRITNPGAESLGYDSDGHEIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVAEWDKLRQRLLDARNKITSAESAVNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVRTENWRPLFVKTETIIAGNAATALVALVFSILTGSALGIIGYGLLMAVTGALIDESLVEKANKFWGIDSVCASYF

SEQ ID NO: 10 - DNA, 1905 bp, Artificial sequence
  Colicin Ia attached to Pheromone peptide from Staphylococcus epidermidis
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatcattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatacgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtattgattccgtttgtgcatcctatttttaa

SEQ ID NO: 11 - protein, 633 aa, Artificial sequence
  Pheromone peptide from Staphylococcus aureus AgrDI attached to Colicin Ia
YSTCDFIMSDPVRITNPGAESLGYDSDGHEIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVAEWDKLRQRLLDARNKITSAESAVNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVRTENWRPLFVKTETIIAGNAATALVALVFSILTGSALGIIGYGLLMAVTGALIDESLVEKANKFWGI

SEQ ID NO: 12 - DNA, 1905 bp, Artificial sequence
  Pheromone peptide from Staphylococcus aureus AgrDI attached to Colicin Ia
atgtattccacctgtgattttataatgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatcattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatacgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtatttaa

SEQ ID NO: 13 - protein, 651 aa, Artificial sequence
  Colicin Ia attached to Pheromone peptide from Staphylococcus epidermidis
SDPVARGITNPGAESLGYDSDGHEIMAVASPIYVASNPPRVASPVFHGTPPAWSSFGNKTIWGGNEWVASPDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVALAEWDKLRQRLLDARNKITSAESAVASNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVARGTENWRPLFVKTETIIAGNAATALVALALVFSILTGSALGIIGYGLLMAVTGALIDESLVEKANKFWGIDSVCASYF

SEQ ID NO: 14 - DNA, 1902 bp, Artificial sequence
  Colicin Ia attached to Pheromone peptide from Staphylococcus epidermidis
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatcattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatacgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtattgactctgtttgcgcttcttacttt

SEQ ID NO: 15 - protein, 660 aa, Artificial sequence
  colicin Ia attached to streptococcus pneumoniae polypeptide pheromone
SDPVARGITNPGAESLGYDSDGHEIMAVASPIYVASNPPRVASPVFHGTPPAWSSFGNKTIWGGNEWVASPDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVALAEWDKLRQRLLDARNKITSAESAVASNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVARGTENWRPLFVKTETIIAGNAATALVALALVFSILTGSALGIIGYGLLMAVTGALIDESLVEKANKFWGIEMRLSKFFRDFILQRKK

SEQ ID NO: 16 - DNA, 1929 bp, Artificial sequence
  colicin Ia attached to streptococcus pneumoniae polypeptide pheromone
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatcattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatacgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtattgagatgaggttgtcaaaattcttccgtgattttattttacaaagaaaaaag

SEQ ID NO: 17 - protein, 650 aa, Artificial sequence
  Colicin Ia linked to E. facaelis pheromone cCF10
SDPVARGITNPGAESLGYDSDGHEIMAVASPIYVASNPPRVASPVFHGTPPAWSSFGNKTIWGGNEWVASPDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVALAEWDKLRQRLLDARNKITSAESAVASNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVARGTENWRPLFVKTETIIAGNAATALVALALVFSILTGSALGIIGYGLLMAVTGALIDESLVEKANKFWGILVTLVFV

SEQ ID NO: 18 - DNA, 1899 bp, Artificial sequence
  Colicin Ia linked to E. facaelis pheromone cCF10
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatcattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatacgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtattcttgtcacgcttgtcttcgtc

SEQ ID NO: 19 - protein, 678 aa, Artificial sequence
  colicin Ia attached to G28-V50/E216-S228 from HBsAg ScFv
SDPVARGITNPGAESLGYDSDGHEIMAVASPIYVASNPPRVASPVFHGTPPAWSSFGNKTIWGGNEWVASPDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVALAEWDKLRQRLLDARNKITSAESAVASNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVARGTENWRPLFVKTETIIAGNAATALVALALVFSILTGSALGIIGYGLLMAVTGALIDESLVEKANKFWGIGFTFSDYYMSWIRQAPGKGLEWVEDEADYYCNSRD

SEQ ID NO: 20 - DNA, 2034 bp, Artificial sequence
  colicin Ia attached to G28-V50/E216-S228 from HBsAg ScFv
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatcattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatacgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtattggattcaccttcagtgactactacatgagctggatccgccaggctccagggaaggggctggagtgggttgaagatgaggctgactattactgtaactcccgggacagcagtggtgaagatgaggctgactattactgtaactcccgggacagcagt

SEQ ID NO: 21 - protein, 656 aa, Artificial sequence
  colicin Ia and the C. albicans mating pheromone
SDPVARGITNPGAESLGYDSDGHEIMAVASPIYVASNPPRVASPVFHGTPPAWSSFGNKTIWGGNEWVASPDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVALAEWDKLRQRLLDARNKITSAESAVASNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVARGTENWRPLFVKTETIIAGNAATALVALALVFSILTGSALGIIGYGLLMAVTGALIDESLVEKANKFWGIGFRLTNFGYFEPG

SEQ ID NO: 22 - DNA, 1917 bp, Artificial sequence
  colicin Ia and the C. albicans mating pheromone
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatcattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatacgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtattggtttccgtctgactagattcggttatttctaacctggc

SEQ ID NO: 23 - protein, 8 aa, S. Aurues
YSTCDFIM

SEQ ID NO: 24 - protein, 11 aa, S. Aurues
GVASNACSSLF

SEQ ID NO: 25 - protein, 8 aa, S. Aurues
YINCDFLL

SEQ ID NO: 26 - protein, 8 aa, S. Aurues
YSTCFFIM

SEQ ID NO: 27 - protein, 8 aa, S. epidermis
DSVCASYF

SEQ ID NO: 28 - protein, 43 aa, S. Pneumoniae
MKNTVKLEQFVALALKEKDLQKIKGGEMRLSKFFRDFILQRKK

SEQ ID NO: 29 - protein, 7 aa, E. Facaelis
LVTLVFV

SEQ ID NO: 30 - protein, 644 aa, E. coli
MSDPVARGITNPGAESLGYDSDGHEIMAVASPIYVASNPPRVASPVFHGTPPAWSSFGNKTIWGGNEWVASPDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVALAEWDKLRQRLLDARNKITSAESAVASNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVARGTENWRPLFVKTETIIAGNAATALVALALVFSILTGSALGIIGYGLLMAVTGALIDESLVEKANKFWGI

SEQ ID NO: 31 - DNA, 1878 bp, E. coli
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatcattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatacgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtatt

SEQ ID NO: 32 - protein, 322 aa, Artificial sequence
  HBV PreS1 scFV linked to a pore-forming region of Colicin Ia
KFTSLADWITEFGKAVARGTENWRPLFVKTETIIAGNAATALVALALVFSILTGSALGIIGYGLLMAVTGALIDESLVEKANKFWGIQMQLVQSGAEVKKPGSSVKVSCKASGGTFSSYAISWVARGQAAGQGLEWMGGIIPKFGTPNYIQKFQGRVTITADKSTTTAYMELKNLRSEDTAVYYCARNGSGGAEYYNGLDVSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLHARVFGGGT

SEQ ID NO: 33 - DNA, 976 bp, Artificial sequence
  HBV PreS1 scFV linked to a pore-forming region of Colicin Ia
aaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtattgattccgtttgtgcatcctatttttaacagatgcagctggtgcagtctggggctgaggtgaagaagcctgggtcctcggtgaaggtctcctgcaaggcttctggaggcaccttcagcagctatgctatcagctgggtgcgacaggccgctggacaagggcttgagtggatgggagggatcatacctaaatttggtacaccaaactacatacagaagttccagggcagagtcacgattaccgcggacaagtccacgaccacagcctacatggagctgaagaacctgagatctgaggacacggccgtatattactgtgcgagaaacgggtcagggggagccgaatactacaacggtttggacgtctccggtggaggcggttcaggcggaggtggctctggtggaggcggtagtcagtctgtgctgactcagccaccctcagcgtctgggacccccgggcagagggtcaccatctcttgttctggaagcagctccaacatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgcatgctcgtgtattcggcggagggacca

SEQ ID NO: 34 - protein, 879 aa, Artificial sequence
  HBV PreS1 scFV linked to Colicin Ia
MSDPVARGITNPGAESLGYDSDGHEIMAVASPIYVASNPPRVASPVFHGTPPAWSSFGNKTIWGGNEWVASPDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVALAEWDKLRQRLLDARNKITSAESAVASNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVARGTENWRPLFVKTETIIAGNAATALVALALVFSILTGSALGIIGYGLLMAVTGALIDESLVEKANKFWGIQMQLVQSGAEVKKPGSSVKVSCKASGGTFSSYAISWVARGQAAGQGLEWMGGIIPKFGTPNYIQKFQGRVTITADKSTTTAYMELKNLRSEDTAVYYCARNGSGGAEYYNGLDVSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLHARVFGGGT

SEQ ID NO: 35 - DNA, 2578 bp, Artificial sequence
  HBV PreS1 scFV linked to Colicin Ia
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatcattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatacgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtattcagatgcagctggtgcagtctggggctgaggtgaagaagcctgggtcctcggtgaaggtctcctgcaaggcttctggaggcaccttcagcagctatgctatcagctgggtgcgacaggccgctggacaagggcttgagtggatgggagggatcatacctaaatttggtacaccaaactacatacagaagttccagggcagagtcacgattaccgcggacaagtccacgaccacagcctacatggagctgaagaacctgagatctgaggacacggccgtatattactgtgcgagaaacgggtcagggggagccgaatactacaacggtttggacgtctccggtggaggcggttcaggcggaggtggctctggtggaggcggtagtcagtctgtgctgactcagccaccctcagcgtctgggacccccgggcagagggtcaccatctcttgttctggaagcagctccaacatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgcatgctcgtgtattcggcggagggacca

SEQ ID NO: 36 - protein, 679 aa, Artificial sequence
  G28 - V50/E217 - S228 of HBsAg ScFV linked to colicin Ia
MSDPVARGITNPGAESLGYDSDGHEIMAVASPIYVASNPPRVASPVFHGTPPAWSSFGNKTIWGGNEWVASPDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVALAEWDKLRQRLLDARNKITSAESAVASNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVARGTENWRPLFVKTETIIAGNAATALVALALVFSILTGSALGIIGYGLLMAVTGALIDESLVEKANKFWGIGFTFSDYYMSWIRQAPGKGLEWVEDEADYYCNSRD

SEQ ID NO: 37 - DNA, 1975 bp, Artificial sequence
  G28 - V50/E217 - S228 of HBsAg ScFV linked to colicin Ia
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatcattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatacgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtattggagtgggttgaagatgaggctgactattactgtaactcccgggacagcagtggtgaagatgaggctgactattactgtaactcccgggacagcagt

SEQ ID NO: 38 - protein, 673 aa, Artificial sequence
  CDRs from HBV PreS-1 ScFv  linked to colicin Ia
MSDPVARGITNPGAESLGYDSDGHEIMAVASPIYVASNPPRVASPVFHGTPPAWSSFGNKTIWGGNEWVASPDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVALAEWDKLRQRLLDARNKITSAESAVASNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVARGTENWRPLFVKTETIIAGNAATALVALALVFSILTGSALGIIGYGLLMAVTGALIDESLVEKANKFWGISYAISWVARGQAAGQGLEWMGAAWDDSLH

SEQ ID NO: 39 - DNA, 1959 bp, Artificial sequence
  CDRs from HBV PreS-1 ScFv  linked to colicin Ia
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatcattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatacgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtattagctatgctatcagctgggtgcgacaggccgctggacaagggcttgagtggatgggagcagcatgggatgacagcctgcat

SEQ ID NO: 40 - protein, 18 aa, Artificial sequence
  HBV PreS-1  ScFv  CDR2 from the heavy chain
GIIPKFGTPNYIQKFQGR

SEQ ID NO: 41 - protein, 8 aa, Artificial sequence
  HBV PreS-1  ScFv  CDR2 from the light chain
YRNNQRPS

SEQ ID NO: 42 - protein, 14 aa, Artificial sequence
  HBV PreS-1  ScFv  CDR1-CDR2 light chain linker
WYQQLPGTAPKLLI

SEQ ID NO: 43 - protein, 683 aa, Artificial sequence
  CDRs from HBV PreS-1  ScFv attached to colicin Ia
SDPVARGITNPGAESLGYDSDGHEIMAVASPIYVASNPPRVASPVFHGTPPAWSSFGNKTIWGGNEWVASPDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVALAEWDKLRQRLLDARNKITSAESAVASNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVARGTENWRPLFVKTETIIAGNAATALVALALVFSILTGSALGIIGYGLLMAVTGALIDESLVEKANKFWGIGIIPKFGTPNYIQKFQGRWYQQLPGTAPKLLIYRNNQRPS

SEQ ID NO: 44 - DNA, 1998 bp, Artificial sequence
  CDRs from HBV PreS-1  ScFv attached to colicin Ia
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatcattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatacgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtattgggatcatacctaaatttggtacaccaaactacatacagaagttccagggcagatggtaccagcagctcccaggaacggcccccaaactcctcatctataggaataatcagcggccctca

SEQ ID NO: 45 - protein, 10 aa, Artificial sequence
  HBV HBsAg ScFv CDR1 from the heavy chain
GFTFSDYYMS

SEQ ID NO: 46 - protein, 12 aa, Artificial sequence
  HBV HBsAg ScFv CDR3 from the light chain
DEADYYCNSRDS

SEQ ID NO: 47 - protein, 14 aa, Artificial sequence
  HBV HBsAg ScFv CDR1-CDR2 linker from the light chain
WIRQAPGKGLEWVS

SEQ ID NO: 48 - protein, 679 aa, Artificial sequence
  CDRs and FR from HBV HBsAg ScFv linked to Colicin Ia
SDPVARGITNPGAESLGYDSDGHEIMAVASPIYVASNPPRVASPVFHGTPPAWSSFGNKTIWGGNEWVASPDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVALAEWDKLRQRLLDARNKITSAESAVASNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVARGTENWRPLFVKTETIIAGNAATALVALALVFSILTGSALGIIGYGLLMAVTGALIDESLVEKANKFWGIGFTFSDYYMSWIRQAPGKGLEWVSDEADYYCNSRDS

SEQ ID NO: 49 - DNA, 1986 bp, Artificial sequence
  CDRs and FR from HBV HBsAg ScFv linked to Colicin Ia
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatcattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatacgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtattggctttaccttcagtgattattatatgtcttggattcgccaggcgccgggcaaaggcctggaatgggtgtccgatgaagcggattattattgcaacagccgcgattcc

SEQ ID NO: 50 - protein, 10 aa, Artificial sequence
  G28 - S37 from HBV HBsAg ScFV
GFTFSDYYMS

SEQ ID NO: 51 - protein, 653 aa, Artificial sequence
  G28 - S37 from HBV HBsAg ScFV linked to Colicin Ia
SDPVARGITNPGAESLGYDSDGHEIMAVASPIYVASNPPRVASPVFHGTPPAWSSFGNKTIWGGNEWVASPDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVALAEWDKLRQRLLDARNKITSAESAVASNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVARGTENWRPLFVKTETIIAGNAATALVALALVFSILTGSALGIIGYGLLMAVTGALIDESLVEKANKFWGIGFTFSDYYMS

SEQ ID NO: 52 - DNA, 1908 bp, Artificial sequence
  G28 - S37 from HBV HBsAg ScFV linked to Colicin Ia
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatcattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatacgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtattggattcaccttcagtgactactacatgagc

SEQ ID NO: 53 - protein, 12 aa, Artificial sequence
  E217 - S228 from HBV HBsAg
DEADYYCNSRDS

SEQ ID NO: 54 - protein, 655 aa, Artificial sequence
  E217 - S228 from HBV HBsAg ScFV linked to Colicin Ia
SDPVARGITNPGAESLGYDSDGHEIMAVASPIYVASNPPRVASPVFHGTPPAWSSFGNKTIWGGNEWVASPDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVALAEWDKLRQRLLDARNKITSAESAVASNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVARGTENWRPLFVKTETIIAGNAATALVALALVFSILTGSALGIIGYGLLMAVTGALIDESLVEKANKFWGIDEADYYCNSRDS

SEQ ID NO: 55 - DNA, 1911 bp, Artificial sequence
  E217 - S228 from HBV HBsAg ScFV linked to Colicin Ia
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatcattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatacgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtattgatgaggctgactattactgtaactcccgggac

SEQ ID NO: 56 - protein, 650 aa, Artificial sequence
  phage m13 peptide linked to colicin Ia
MSPVARGITNPGAESLGYDSDGHEIMAVASPIYVASNPPRVASPVFHGTPPAWSSFGNKTIWGGNEWVASPDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVALAEWDKLRQRLLDARNKITSAESAVASNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVARGTENWRPLFVKTETIIAGNAATALVALALVFSILTGSALGIIGYGLLMAVTGALIDESLVEKANKFWGITLTTKLY

SEQ ID NO: 57 - protein, 653 aa, Artificial sequence
  phage m13 peptide linked to colicin Ia
MSDPVARGITNPGAESLGYDSDGHEIMAVASPIYVASNPPRVASPVFHGTPPAWSSFGNKTIWGGNEWVASPDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVALAEWDKLRQRLLDARNKITSAESAVASNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVARGTENWRPLFVKTETIIAGNAATALVALALVFSILTGSALGIIGYGLLMAVTGALIDESLVEKANKFWGICTLTTKLYC

SEQ ID NO: 58 - DNA, 1899 bp, Artificial sequence
  phage m13 peptide linked to colicin Ia
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatcattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatacgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtattactctcactaccaaactctac

SEQ ID NO: 59 - DNA, 1904 bp, Artificial sequence
  phage m13 peptide linked to colicin Ia
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatcattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatacgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtatttgtactctcactaccaactatactgt

SEQ ID NO: 60 - protein, 13 aa, Candida albicans
GFRLTNFGYFEPG

SEQ ID NO: 61 - protein, 657 aa, Artificial sequence
  Candida albicans alpha mating pheromone attached to colicin
GFRLTNFGYFEPGMSDPVARGITNPGAESLGYDSDGHEIMAVASPIYVASNPPRVASPVFHGTPPAWSSFGNKTIWGGNEWVASPDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVALAEWDKLRQRLLDARNKITSAESAVASNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVARGTENWRPLFVKTETIIAGNAATALVALALVFSILTGSALGIIGYGLLMAVTGALIDESLVEKANKFWGI

SEQ ID NO: 62 - DNA, 1917 bp, Artificial sequence
  Candida albicans alpha mating pheromone attached to colicin
ggtttccgactgactcgtttcggttattttgaacctggtatgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatcattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatacgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtatt